(19) United States
(12) Patent Application Publication
(10) Pub. No.: US 2023/0372474 A1
(43) Pub. Date: Nov. 23, 2023

(54) RECOMBINANT HVT AND USES THEREOF

(71) Applicant: Ceva Sante Animale, Liboume (FR)

(72) Inventors: Motoyuki Esaki, Yokohama-city (JP); Vilmos Palya, Budapest (HU); Timea Tatar, Budapest (HU); Zoltan Penzes, Budapest (HU)

(21) Appl. No.: 18/031,102

(22) PCT Filed: Oct. 14, 2021
(86) PCT No.: PCT/EP2021/078432

§ 371 Date: Apr. 10, 2023

(30) Foreign Application Priority Data
Oct. 15, 2020 (EP) 20306212.0

(51) Int. Cl. A61K 39/245; C12N 7/00; C12N 15/86; A61P 31/22

(52) U.S. Cl. CPC A61K 39/245; C12N 7/00; C12N 15/86; A61P 31/22; C12N 2710/16034; C12N 2710/16043; C12N 2710/16021; C12N 2760/18122; C12N 2760/16122; A61K 2039/5256

(57) ABSTRACT

The present invention relates to recombinant herpes viruses comprising recombinant nucleotide sequences encoding at least two different antigens. The invention is particularly suited for producing vaccines to immunize avian species against avian pathogen(s).

### FIELD OF THE INVENTION

The present invention relates to recombinant avian herpes viruses encoding different antigens, and the uses thereof. The invention is suited for producing vaccines to immunize avian species against avian pathogen(s).

### BACKGROUND OF THE INVENTION

Poultry meat and eggs are important food sources, whose consumption increases continually due to the growth of the human population and their great quality-price ratio. In order to ensure poultry health as well as food safety and security, poultry vaccine technology has become a worldwide concern.

Viral vectors expressing pathogen proteins are commonly used as poultry vaccines against targeted pathogens. Vaccines including such viral vectors induce expression of foreign pathogen proteins within infected hosts, which may lead to protective immunity.

Many different classes of viruses have been investigated as candidate vectors for vaccination of avians, such as adenoviruses, AAVs, fowlpox viruses, herpes viruses, and the like.

Three types of herpes viruses have been determined, MDV1, MDV2 and MDV3 (also known as herpes virus of turkey (HVT)). High similarity exists between said viruses (see Kingham et al., Journal of General Virology (2001) 82, 1123-1135) and they all have been used to prepare recombinant viruses in which a foreign gene derived from a pathogen has been integrated, for use as a vaccine in avians, particularly poultry, such as chicken.

Although such vaccine preparations provide efficient results to vaccinate avian species against many diseases, competition and immunosuppression between pathogens can occur when avians are injected with two or more recombinant herpes viruses, each encoding a different antigen.

In order to overcome such interference and also to facilitate vaccination against multiple diseases, various attempts have been made to produce multivalent herpes viruses encoding several antigens.

First studies inserted several genes in a single cloning site in the genome of the herpes virus (see e.g., EP1026246). However, such constructs either did not provide the required level of protective immunity or turned out to be unstable, all or part of the foreign genes being deleted during repeating passages in culture cells.

WO2013/144355 and WO2020/127964 report stable herpes viruses encoding multiple foreign antigens obtained using combinations of cloning sites located in non-coding regions of the viral genome.

WO2013/057236, WO2013/082327 and WO2013/082317 report another approach in the design of multivalent HVT, by cloning at least one gene within the US2 coding sequence of herpes viruses.

Considering the number of pathogens and species, there is a need in the art for further recombinant multivalent herpes viruses which can stably express multiple genes in vivo and are suitable for vaccination of avians, particularly poultry.

### SUMMARY OF THE INVENTION

The present invention provides recombinant avian herpes viruses comprising at least two recombinant nucleotide sequences in at least two separate locations of the viral genome.

More specifically, the invention provides recombinant herpes viruses of turkey (HVT) comprising (i) a nucleotide sequence encoding a F protein of Newcastle Disease Virus, or an immunogenic fragment or variant thereof, inserted into a first insertion site of the viral genome; and (ii) a nucleotide sequence encoding an Hemagglutinin (HA) protein of a subtype H9 Avian Influenza Virus, or an immunogenic fragment or variant thereof, inserted into a second insertion site of the viral genome, the first and second insertion sites being located in different non-coding regions of the viral genome selected from the non-coding region between UL45 and UL46, and the non-coding region between SORF3 and US2.

The invention also relates to a nucleic acid comprising the genome of a recombinant HVT as defined above, and to a vector (such as a plasmid) containing such a nucleic acid.

The invention also relates to novel antigens and nucleic acid molecules encoding the same.

The invention further relates to a cell containing a recombinant HVT or a nucleic acid or vector as defined above.

A further object of the invention is a composition comprising a recombinant HVT as defined above and a suitable excipient or diluent.

A further object of the invention is a composition comprising a nucleic acid or a cell as defined above and a suitable excipient or diluent.

Another object of the invention resides in a vaccine which comprises an effective immunizing amount of a recombinant HVT, nucleic acid and/or cell, as defined above.

A further object of the invention resides in a recombinant HVT, nucleic acid or cell as defined above, for use for immunizing an avian, such as poultry, against Newcastle Disease Virus (NDV) and Avian Influenza Virus (AIV), and/or an associated disease.

A further object of the invention resides in a recombinant HVT, nucleic acid or cell as defined above, for use for protecting an avian, such as poultry, against a disease caused by NDV and AIV.

A further object of the invention resides in a vaccine as defined above, for use for vaccinating an avian, such as poultry, against NDV and AIV.

A further object of the invention resides in a method for vaccinating an avian comprising administering to the avian a composition or vaccine or virus as defined above.

A further object of the invention resides in a method for inducing an immune response to an antigen in an avian comprising administering to the avian a composition or vaccine or virus as defined above.

The invention also provides a vaccination kit for immunizing an avian which comprises the following components:
a. an effective amount of a composition or vaccine as defined above, and
b. means for administering said composition or vaccine to said avian.

The invention may be used in any avian, for vaccination against NDV and/or AIV, and/or an associated disorder or condition. It is particularly suited to vaccinate poultry, such as chicken.

### BRIEF DESCRIPTION OF THE DRAWINGS

FIG. 1 illustrates schematic diagrams of recombinant bivalent HVT constructs with NDV F gene and AIV HA-H9 gene according to the invention (FW205, FW206, FW208, FW209, FW247, FW248, FW249, FW250, FW251, FW252), and recombinant monovalent HVT constructs with NDV F gene (FW168) or AIV HA-H9 gene (FW202, FW204).

FIG. 2 shows results of immunofluorescence assay demonstrating expression of (A) NDV F protein (green fluorescence), (B) AIV HA-H9 protein (red fluorescence) and (C) merged (yellow), by rHVT/ND-H9 constructs.

FIG. 3 shows results of western blot assay detecting expression of NDV F protein by constructs FW168, FW205, FW206, FW208 and FW209.

FIG. 4 shows results of western blot assay detecting expression of AIV HA-H9 protein by constructs FW205, FW206, FW208 and FW209.

FIG. 5 shows results of western blot assay detecting expression of NDV F protein by constructs FW247, FW248, FW249, FW250, FW251, FW252, FW168, and FW206.

FIG. 6 shows results of western blot assay detecting expression of AIV HA-H9 protein by constructs FW247, FW248, FW249, FW250, FW251, FW252, FW204, and FW206.

FIG. 7 shows NDV ELISA titers in chickens vaccinated with constructs FW205, FW206, and FW208, using a commercial NDV ELISA kit.

FIG. 8 shows AIV H9 HI titers in chickens vaccinated with constructs FW205 and FW208 (FIG. 8A), and FW206 (FIG. 8B).

FIG. 9 shows AIV load in tracheal swabs of chickens vaccinated with constructs FW205, FW206 and FW208, after challenge with AIV of H9 subtype.

FIG. 10 shows air sac lesion scores of chickens vaccinated with constructs FW205, FW206 and FW208, after challenge with AIV of H9 subtype.

FIG. 11 shows NDV ELISA titers in chickens vaccinated with constructs FW247, FW248, FW249, FW250, FW251, and FW252, using a commercial NDV ELISA kit.

FIG. 12 shows AIV H9 HI titers in chickens vaccinated with constructs FW247, FW248, FW249, FW250, FW251, and FW252, using a commercial NDV ELISA kit.

### DETAILED DESCRIPTION OF THE INVENTION

The present invention generally relates to recombinant avian herpes viruses containing multiple recombinant nucleotide sequences, their manufacture, compositions comprising the same, and the uses thereof. The invention also provides novel antigens suitable for generating a potent immune response against AIV.

### Definitions

The present disclosure will be best understood by reference to the following definitions:

The term “recombinant”, in relation to a herpes virus, refers to a herpes virus the genome of which has been modified by insertion of at least one nucleotide sequence (e.g., DNA, such as a gene) which is not found naturally in the genome of the herpes virus, or which is found naturally in said genome but in a different form or at a different position. It will be understood that the recombinant herpes virus can be manufactured by a variety of methods such as recombinant DNA technology as described therein and, once made, can be reproduced without further use of recombinant DNA technology.

In the present description, the terms “nucleic acid”, “nucleic sequence,” and “nucleotide sequence” are used interchangeably and refer to a nucleic acid molecule having a determined sequence, which may be deoxyribonucleotides and/or ribonucleotides. The nucleotide sequence may be first prepared by e.g., recombinant, enzymatic and/or chemical techniques, and subsequently replicated in a host cell or an in vitro system. A nucleotide sequence preferentially comprises an open reading frame encoding a molecule (e.g. a peptide or protein). The nucleotide sequence may contain additional sequences such as a promoter, a transcription terminator, a signal peptide, an IRES, etc.

In the present description, the terms “polypeptide”, “peptide,” and “protein” are used interchangeably and refer to any molecule comprising a polymer of at least 2 consecutive amino acids.

The term “non-coding region” is well known in the art and refers to any region of a viral genome which does not encode a protein. The non-coding region between UL45 (HVT053) and UL46 (HVT054) designates typically a region starting immediately 3′ of the STOP codon of UL45 and ending immediately 5′ of the STOP codon of UL46 (since both ORFs are in opposite orientation). The non-coding region between SORF3 (HVT087) and US2 (HVT088) designates typically a region starting immediately 3′ of the START codon of SORF3 and ending immediately 5′ of the STOP codon of US2.

An “immunogenic fragment” of an antigen designates any fragment which can elicit an immune response, preferably any fragment which contains an epitope, preferably an antigen-specific epitope. Immunogenic fragments generally contain from 5 to 50 consecutive amino acid residues of an antigen, such as from 5 to 40, or from 10 to 40, or 10-30, 10-25, or 10-20. Examples of fragments of a native F protein include any fragment of from 10 to 40 consecutive amino acids of SEQ ID NO: 1.

As used herein, the term “variant” refers to a modified form of a reference antigen or fragment, which retains an immunogenic property thereof. Generally, variants are overall similar and, in many regions, identical to the reference antigen or fragment. For instance, a variant may exhibit at least about 70%, at least about 80%, at least about 90%, at least about 95%, at least about 96%, at least about 97%, at least about 98%, or at least about 99% sequence identity compared to the reference antigen or fragment. Variants particularly designate antigens with 1, 2, 3, 4 or 5 modified amino acid residues as compared to the reference sequence. Modifications include amino acid deletion(s), substitution(s), and/or addition(s). Variants shall retain an immunogenic property of the reference sequence, such as an ability to induce an immune response against the reference sequence or pathogen. Examples of variants of a native F protein include any protein comprising, or consisting of SEQ ID NO: 1, with 1, 2 or 3 amino acid substitutions. Examples of variants of a fragment include a protein consisting of from 10 to 40 consecutive amino acids of SEQ ID NO: 1, with 1, 2 or 3 amino acid substitutions.

The term “avian species” is intended to encompass all kinds of avians such as birds of the class of Aves, i.e., vertebrate animals which are feathered, winged, bipedal, endothermic and egg-laying. In the context of the invention, avians or avian species refer more particularly to birds with economical and/or agronomical interests, such as poultry, (such as chickens and turkeys), waterfowl poultry (such as ducks and geese) and ornamental birds (such as swans and psittacines).

The term “vaccine” as used herein designates an agent which may be used to cause, stimulate or amplify an immune response in an organism.

Recombinant HVT

The invention relates to recombinant HVT containing multiple foreign genes in particular locations. More specifically, the invention relates to recombinant HVTs (rHVTs) comprising:
(i) a nucleotide sequence encoding a F protein of Newcastle Disease Virus, or an immunogenic fragment or variant thereof, inserted into a first insertion site of the viral genome; and
(ii) a nucleotide sequence encoding a Hemagglutinin (HA) protein of a subtype H9 Avian Influenza Virus, or an immunogenic fragment or variant thereof, inserted into a second insertion site of the viral genome;
the first and second insertion sites being located in different non-coding regions of the viral genome selected from the non-coding region between UL45 and UL46 and the non-coding region between SORF3 and US2.

In a particular embodiment, the invention relates to a rHVT comprising:
(i) a nucleotide sequence encoding a F protein of Newcastle Disease Virus, or an immunogenic fragment or variant thereof, inserted into a non-coding region of the viral genome located between UL45 and UL46; and
(ii) a nucleotide sequence encoding a Hemagglutinin (HA) protein of a subtype H9 avian influenza virus, or an immunogenic fragment or variant thereof, inserted into a non-coding region of the viral genome located between SORF3 and US2.

In a further particular embodiment, the invention relates to a rHVT comprising:
(i) a nucleotide sequence encoding a F protein of Newcastle Disease Virus, or an immunogenic fragment or variant thereof, inserted into a non-coding region of the viral genome located between SORF3 and US2; and
(ii) a nucleotide sequence encoding a Hemagglutinin (HA) protein of a subtype H9 avian influenza virus, or an immunogenic fragment or variant thereof, inserted into a non-coding region of the viral genome located between UL45 and UL46.

As shown in the examples, such constructs are genetically stable over at least 10, preferably at least 15, more preferably at least 20 passages in CEF cells. Such constructs also provide stable co-expression of antigens over at least 10, preferably at least 15, more preferably at least 20 passages in CEF cells. They can confer strong and long-lasting expression of the genes in vivo, sufficient to procure high protective immunity.

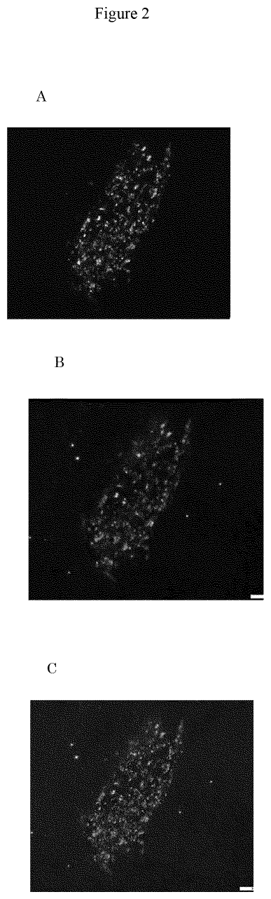

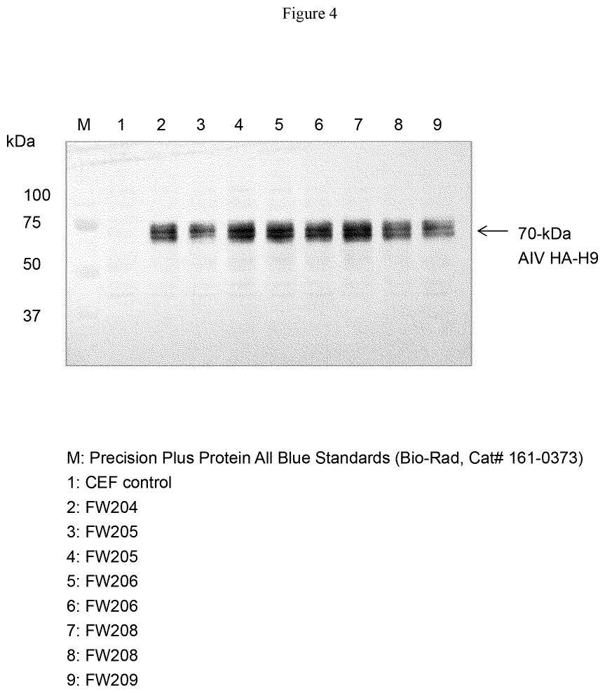

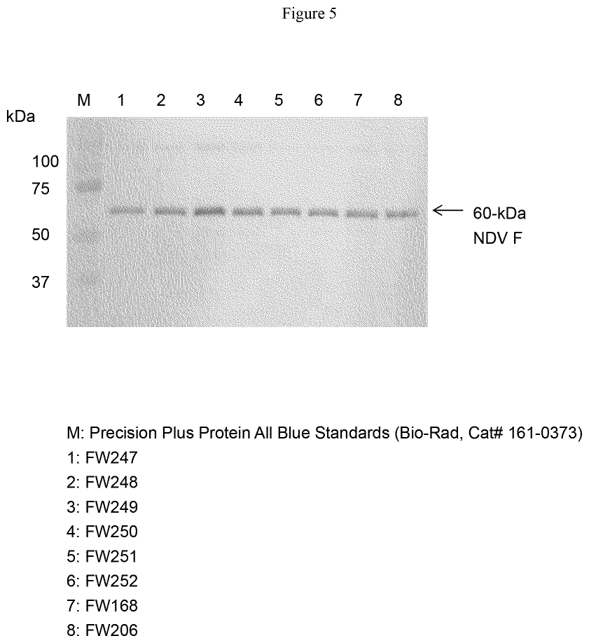

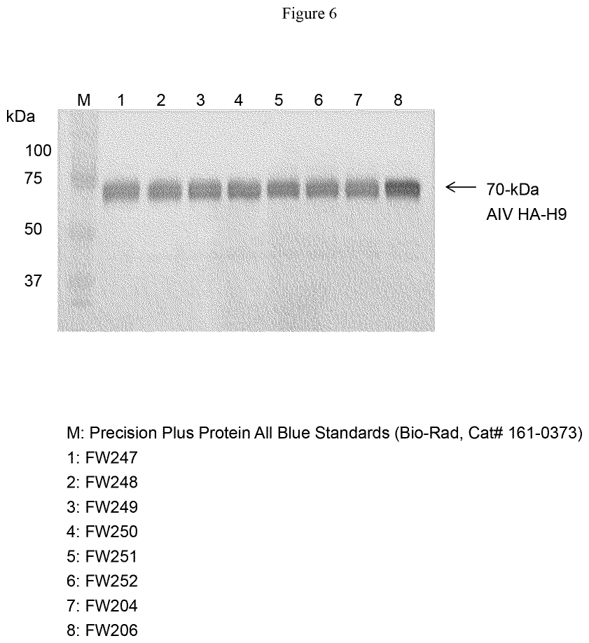

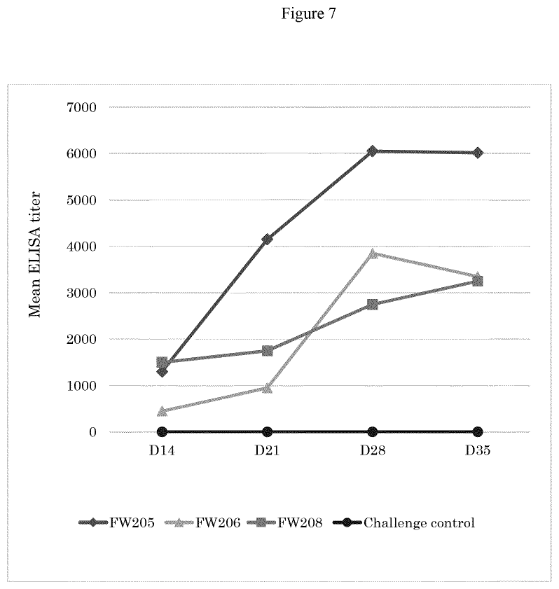

More particularly, the inventors have demonstrated that the claimed rHVTs correctly express both NDV F and AIV HA-H9 antigens (FIGS. 2-6). The inventors have also demonstrated that the claimed rHVTs efficiently induce antibodies against NDV F and AIV HA-H9 (FIGS. 7, 8, 11 and 12), with high HI titers. The data in the present application further show that SPF chickens vaccinated with bivalent constructs of the invention are very efficiently protected (e.g., up to 100%) against Newcastle Disease (ND) and AIV after challenge, and that all vaccinated groups have lower AIV viral load as compared to the control group.

The claimed rHVTs thus confer very efficient clinical protection against challenge with NDV and AIV. The invention thus provides novel effective constructs that can be used to protect avians against highly relevant pathogens and associated disorders.

Recombinant HVTs of the invention may be prepared from any HVT, preferably non-pathogenic HVT. An example of a non-pathogenic strain of HVT (MDV3) suitable for use in the invention is the FC126 strain. The genomic sequence of the FC126 strain is available in the art (Afonso et al., supra; Kingham et al. supra). Another suitable HVT strain is the PB1 strain, for instance. Any other non-pathogenic strain is suitable as well.

The location of the targeted non-coding regions in the viral genome can be easily identified by the skilled person using the teachings of the present application, common knowledge and sequence information available in the literature. For instance, Kingham et al. supra, reports the nucleotide sequence of the FC126 reference strain, as well as the location of most ORFs within said genome.

By reference to a FC126 complete genome (GenBank: AF291866.1), a non-coding region between UL45 and UL46 corresponds to nucleotides 95323-95443 of the HVT genome, and a non-coding region between SORF3 and US2 corresponds to nucleotides 139867-140064 of the HVT genome. Cloning at any position within said regions is suitable for the present invention.

NDV F Protein

As indicated, the claimed rHVTs contain a recombinant nucleotide sequence which encodes a F protein of NDV, or an immunogenic fragment or variant thereof.

NDV F protein is a F protein of Newcastle Disease virus (also called Avian Paramyxovirus type 1 virus) being a class I viral membrane fusion (F) glycoprotein which mediates the penetration of the cellular membrane during viral entry into cells. NDV F is a known antigen of NDV. The amino acid sequence of a native NDV F protein is well known and published, such as under No. AAU89279, ABA39232 and AAA46643, as well as any naturally-occurring variants thereof (polymorphisms, splicing variants, etc.). An exemplary sequence is provided as SEQ ID NO: 1 (protein) and SEQ ID NO: 14 (nucleic acid).

The protein encoded by the claimed rHVT may be any native NDV F protein, or any immunogenic fragment or variant thereof which can induce an anti-NDV immune response.

Examples of fragments of a native F protein include any fragment of from 10 to 40 consecutive amino acids of SEQ ID NO: 1. Examples of variants of a native F protein include any protein comprising, or consisting of SEQ ID NO: 1, with 1, 2 or 3 amino acid substitutions. Examples of variants of a fragment include a protein consisting of from 10 to 40 consecutive amino acids of SEQ ID NO: 1, with 1, 2 or 3 amino acid substitutions.

AIV HA Protein

As indicated, the claimed rHVT contain a recombinant nucleotide sequence which encodes a surface protein hemagglutinin (HA) of a subtype H9 avian influenza virus, or an immunogenic fragment or variant thereof.

Influenza virus A is classified to subtypes based on serologic reactions of the HA surface protein. Serologic subtyping of HA is done by the hemagglutinin inhibition test. Sixteen subtypes of HA, HA1 to HA16, are recognized for AIV (David E. Swayne, David L. Suarez, and Leslie D. Simes. (2013). Influenza. In David E. Swayne (Eds.). Diseases of Poultry, Thirteenth Edition (pp. 181-218)). Any AIV can be easily classified in any such subtype following the above technique and common knowledge. Subtype H9 further includes a specific subclass designated H9N2. Example of subtype H9 AIV strains include A/turkey/Wisconsin/1/1966(H9N2), A/Quail/Hong Kong/G1/1997(H9N2), and A/duck/Hong Kong/Y439/1997(H9N2). Preferably, the HA protein is from a H9N2 subtype AIV.

The HA protein may be any native HA protein of a subtype H9 AIV, preferably, of a H9N2 subtype AIV.

Alternatively, the claimed constructs may encode an immunogenic fragment or variant (as defined above) of a surface protein hemagglutinin (HA) of a H9 subtype AIV, preferably of a H9N2 subtype AIV, which can induce an anti-AIV immune response. In this regard, as detailed in the experimental part, the inventors have designed and synthesized optimized H9 HA antigens, with potent immunogenicity and cross reactivity. Such antigens are disclosed as H9-CS (SEQ ID NO: 2), H9-CNn1 (SEQ ID NO: 3), H9-CNn2 (SEQ ID NO: 4), H9-CNn3 (SEQ ID NO: 5), H9-CNn4 (SEQ ID NO: 6) and H9-CNn5 (SEQ ID NO: 7). Said sequences and antigens represent particular objects of the invention, as well as any vector containing the same and the uses thereof.

In this regard, the invention relates to a polypeptide comprising, consisting essentially of, or consisting of, an amino acid sequence selected from SEQ ID NOs: 2-7 and any polypeptide having at least 97% amino acid sequence identity to anyone of SEQ ID NOs: 2-7 over the entire length thereof, preferably at least 98%, even more preferably at least 99%. Amino acid sequence identity may be determined using any known technique or computer program, such as e.g., BLAST.

A specific object of the invention is a polypeptide comprising, consisting essentially of, or consisting of, an amino acid sequence SEQ ID NO: 2.

Another specific object of the invention is a polypeptide comprising, consisting essentially of, or consisting of, an amino acid sequence SEQ ID NO: 3.

Another specific object of the invention is a polypeptide comprising, consisting essentially of, or consisting of, an amino acid sequence SEQ ID NO: 4.

Another specific object of the invention is a polypeptide comprising, consisting essentially of, or consisting of, an amino acid sequence SEQ ID NO: 5.

Another specific object of the invention is a polypeptide comprising, consisting essentially of, or consisting of, an amino acid sequence SEQ ID NO: 6.

Another specific object of the invention is a polypeptide comprising, consisting essentially of, or consisting of, an amino acid sequence SEQ ID NO: 7.

The invention also relates to a chimeric molecule comprising a polypeptide as above conjugated to a moiety (which may be a polypeptide).

The invention also relates to a nucleic acid encoding a polypeptide as defined above, as well as any vector or cell containing such nucleic acid. Preferred nucleic acid molecules of the invention comprise, consist essentially of, or consist of a sequence selected from anyone of SEQ ID NOs: 8-13. The nucleic acids may be conjugated to regulatory sequences (such as a promoter and/or terminator), and/or included in any cloning or expression vector (such as a plasmid, virus, BAC, etc.).

Other Recombinant Sequences

The recombinant HVT according to the invention may further comprise one or more additional sequence(s) encoding, for example, one or more antigens, cytokines, hormones, co-stimulatory factors, adjuvants, etc.

The recombinant nucleotide sequences inserted in the genome may be in any orientation.

Promoter

The inserted nucleic acid sequences may contain (or be operably linked to) regulatory sequences, such as a promoter and/or a terminator. The promoter used may be either a synthetic or natural, endogenous or heterologous promoter. Any promoter may in principle be used, as long as it can effectively function in the target cells or host. In this regard, the promoter may be eukaryotic, prokaryotic, viral or synthetic promoter, capable of directing gene transcription in avian cells in the context of a multivalent vector. Also, each inserted nucleic acid sequence may contain a promoter, which may be the same or different from each other. In a particular embodiment, each inserted nucleic acid sequence contains a different promoter.

Preferentially, the promoter used for each inserted nucleic acid sequence is selected from a Pec promoter, a Cytomegalovirus (CMV) immediate early 1 (ie1) promoter, particularly a Murine Cytomegalovirus (Mcmv) ie1 promoter or a Human Cytomegalovirus (Hcmv) promoter, a chicken beta-actin (Bac) promoter, a Simian virus 40 (SV40) promoter, and a Rous Sarcoma virus (RSV) promoter, or any fragments thereof which retain a promoter activity.

Preferentially, the NDV F coding sequence and the AIV HA coding sequence are under the control of a different promoter.

In a preferred embodiment, one coding sequence in a rHVT of the invention is linked to a Pec promoter.

In another preferred embodiment, one coding sequence in a rHVT of the invention is linked to a CMV ie1 promoter, particularly a Murine Cytomegalovirus (Mcmv) ie1 promoter or a Human Cytomegalovirus (Hcmv) promoter.

A nucleic acid sequence of a Pec promoter is shown in SEQ ID NO: 15, and a sequence of a Mcmv ie1 promoter is shown in SEQ ID NO: 16. It should be noted that variants of such sequences encoding functional promoters are known and/or can be designed/tested by the skilled artisan, for use in the instant invention.

In a preferred embodiment, the recombinant nucleotide sequence inserted into the non-coding region located between UL45 and UL46 contains a Pec promoter, and the recombinant nucleotide sequence inserted into the non-coding region located between SORF3 and US2 contains a CMV IE1 promoter, particularly a Mcmv ie1 promoter. The results obtained by the inventors show such promoters are particularly efficient when positioned in said cloning sites, in the context of a multivalent vector of the invention.

In another preferred embodiment, the foreign gene inserted into the non-coding region located between UL45 and UL46 contains a CMV IE1 promoter, particularly a Mcmv ie1 promoter, and the recombinant nucleotide sequence inserted into the non-coding region located between SORF3 and US2 contains a Pec promoter. The results obtained by the inventors show such promoters are also particularly efficient when positioned in said cloning sites, in the context of a multivalent vector of the invention.

Preferably, a recombinant HVT of the invention comprises (i) a nucleotide sequence encoding a F protein of Newcastle Disease Virus, or an immunogenic fragment or variant thereof, inserted into the non-coding region between UL45 and UL46 under control of a Pec promoter; and (ii) a nucleotide sequence encoding an Hemagglutinin (HA) protein of a subtype H9 avian influenza virus, or an immunogenic fragment or variant thereof, inserted into the non-coding region between SORF3 and US2 under control of a CMV IE1 promoter, preferably a Mcmv ie1 promoter.

In another preferred embodiment, a recombinant HVT of the invention comprises (i) a nucleotide sequence encoding an Hemagglutinin (HA) protein of a subtype H9 avian influenza virus, or an immunogenic fragment or variant thereof, inserted into the non-coding region between UL45 and UL46 under control of a CMV IE1 promoter, preferably a Mcmv ie1 promoter, and (ii) a nucleotide sequence encoding a F protein of Newcastle Disease Virus, or an immunogenic fragment or variant thereof, inserted into the non-coding region between SORF3 and US2 under control of a Pec promoter.

In another embodiment, a recombinant HVT according to the invention comprises (i) a nucleotide sequence encoding a F protein of Newcastle Disease Virus, or an immunogenic fragment or variant thereof, inserted into the non-coding region between UL45 and UL46 under control of a Pec promoter; and (ii) a nucleotide sequence encoding an Hemagglutinin (HA) protein of a subtype H9 avian influenza virus, or an immunogenic fragment or variant thereof, inserted into the non-coding region between SORF3 and US2 under control of a CMV IE1 promoter, preferably a Hcmv promoter.

Method of Construction

The recombinant HVT of the invention may be prepared using techniques known per se in the art, such as recombinant technology, homologous recombination, site-specific insertion, mutagenesis, and the like.

Gene cloning and plasmid construction are well known to one person of ordinary skill in the art and may be essentially performed by standard molecular biology techniques (Molecular Cloning: A Laboratory Manual. 4th Edition, Cold Spring Harbor Laboratory Press, Woodbury, N.Y. 2012).

Herpes viruses may be propagated in any suitable host cell and media. The host and the conditions for propagating herpes viruses include, for instance, cells derived from chicken such as CEF (chick embryo fibroblast), chicken kidney cells, and the like. Such cells may be cultured in a culture medium such as Eagle's MEM, Leibowitz-L-15/McCoy 5A (1:1 mixture) culture medium at about 37° C. for 3 to 4 days.

Genomic DNA may be extracted from virus-infected cells according to any conventional method. In particular, after proteins are denatured in a lysis buffer and removed, DNA may be extracted with phenol and ethanol.

Typically, recombinant viruses may be prepared by homologous recombination between a viral genome and a construct (e.g., a plasmid) comprising the recombinant nucleotide sequence or nucleic acid to be inserted, flanked by nucleotides from the insertion site allowing recombination. Briefly, a sequence containing a targeted region is first cloned into a plasmid, or other suitable vector. Examples of plasmids comprise pBR322, pBR325, pBR327, pBR328, pUC18, pUC19, pUC7, pUC8, and pUC9, examples of phages comprise lambda phage and M13 phage, and example of cosmids comprises pHC79. The cloned region should preferably be of sufficient length so that, upon insertion of the foreign gene, the sequences which flank the foreign gene are of appropriate length so as to allow in vitro homologous recombination with the viral genome. Preferably, each flanking sequence shall have at least approximately 50 nucleotides in length.

In order to insert one or more recombinant nucleotide sequence(s) into the targeted region, mutation may be carried out at a specific site of the region to create a cleavage site for a restriction enzyme. A method of carrying out mutation may be a conventional method, and a method commonly used by a person skilled in the art such as in vitro mutagenesis and PCR can be used. Thus, in the PCR method, a mutation such as the deletion, replacement, or addition of 1 to 2 nucleotides in the PCR primer is carried out, and the primer is then used to create a mutation. Alternatively, naturally existing restriction sites may be used. The foreign gene (and promoter) is then inserted into the insertion site of the viral genome in the plasmid.

The resulting plasmid may be introduced into an HVT-infected cell or HVT genome-transfected cells using any suitable technique (e.g., electroporation, calcium phosphate, a lipofectin-based method or the like). When the amount of the plasmid to be introduced is in the range of 0.1 to 1000 μg, the efficiency of generation of recombinant viruses by recombination between the homologous regions of HVT genome and the plasmid becomes high in cells. This results in a recombination event between the plasmid and the viral genome, leading to insertion of the recombinant nucleotide sequence into the virus.

The resulting recombinant virus may be selected genotypically or phenotypically using known techniques of selection, e.g., by hybridization, detecting enzyme activity encoded by a gene co-integrated along with the recombinant nucleic acid sequences or detecting the antigenic peptide expressed by the recombinant herpes virus immunologically. The selected recombinant herpes virus can be cultured on a large scale in cell culture. Once created, the virus may be propagated in suitable cells.

### Preferred Embodiments

The following recombinant HVT are preferred specific embodiments of the invention. As shown in the examples, they allow strong immune response in vivo against antigens encoded by each recombinant nucleotide sequence.

Particularly preferred recombinant HVT (rHVT) of the invention comprises (i) a nucleotide sequence encoding a F protein of Newcastle Disease Virus, or an immunogenic fragment or variant thereof, inserted into the non-coding region between UL45 and UL46 under control of a Pec promoter; and (ii) a nucleotide sequence encoding an Hemagglutinin (HA) protein of a subtype H9 avian influenza virus, or an immunogenic fragment or variant thereof, inserted into the non-coding region between SORF3 and US2 under control of a Mcmv ie1 promoter. Preferably, such a recombinant rHVT is selected from the following bivalent constructs, as described in the experimental data:
. HVT/45-46 PecF/87-88 Mcmv ie1 H9-CS (FW205),
. HVT/45-46 PecF/87-88 Mcmv ie1 H9-CN (FW206),
. HVT/45-46 PecF/87-88 Mcmv ie1 H9-CNn1 (FW247),
. HVT/45-46 PecF/87-88 Mcmv ie1 H9-CNn2 (FW248),
. HVT/45-46 PecF/87-88 Mcmv ie1 H9-CNn3 (FW249),
. HVT/45-46 PecF/87-88 Mcmv ie1 H9-CNn4 (FW250), and
. HVT/45-46 PecF/87-88 Mcmv ie1 H9-CNn5 (FW251).

In a preferred embodiment, a rHVT according to the invention encodes a HA antigen comprising, or consisting essentially of, or consisting of a sequence selected from anyone of SEQ ID NOs: 2-7.

In a preferred embodiment, a rHVT according to the invention encodes a F antigen comprising, or consisting essentially of, or consisting of SEQ ID NO: 1 or a naturally-occurring variant thereof.

In a preferred embodiment, a rHVT according to the invention contains a nucleic acid encoding a F antigen comprising, or consisting essentially of, or consisting of SEQ ID NO: 14.

In a preferred embodiment, a rHVT according to the invention is HVT/45-46 PecF/87-88 Mcmv ie1 H9-CS (FW205), comprising a consensus sequence of the hemagglutinin gene of avian influenza virus H9 subtype (H9-CS) of SEQ ID NO: 8.

In another preferred embodiment, a rHVT according to the invention is HVT/45-46 PecF/87-88 Mcmv ie1 H9-CNn3 (FW249), comprising an artificially-designed hemagglutinin gene of avian influenza virus H9 subtype (H9-CNn3) of SEQ ID NO: 11.

In other preferred embodiments, a rHVT according to the invention is selected from the following bivalent constructs:
. HVT/45-46 PecF/87-88 Mcmv ie1 H9-CNn1 (FW247), comprising an artificially-designed
hemagglutinin gene of avian influenza virus H9 subtype (H9-CNn1) of SEQ ID NO: 9,
. HVT/45-46 PecF/87-88 Mcmv ie1 H9-CNn2 (FW248), comprising an artificially-designed
hemagglutinin gene of avian influenza virus H9 subtype (H9-CNn2) of SEQ ID NO: 10,
. HVT/45-46 PecF/87-88 Mcmv ie1 H9-CNn4 (FW250), comprising an artificially-designed
hemagglutinin gene of avian influenza virus H9 subtype (H9-CNn4) of SEQ ID NO: 12, and
. HVT/45-46 PecF/87-88 Mcmv ie1 H9-CNn5 (FW251), comprising an artificially-designed hemagglutinin gene of avian influenza virus H9 subtype (H9-CNn5) of SEQ ID NO: 13.

Another preferred rHVT of the invention comprises (i) a nucleotide sequence encoding an Hemagglutinin (HA) protein of a subtype H9 avian influenza virus, or an immunogenic fragment or variant thereof, inserted into the non-coding region between UL45 and UL46 under control of a Mcmv ie1 promoter, and (ii) a nucleotide sequence encoding a F protein of Newcastle Disease Virus, or an immunogenic fragment or variant thereof, inserted into the non-coding region between SORF3 and US2 under control of a Pec promoter. Preferably, such a rHVT is selected from the following bivalent constructs, as described in the experimental data:
. HVT/45-46 Mcmv ie1 H9-CS/87-88 PecF (FW208), and
. HVT/45-46 Mcmv ie1 H9-CN/87-88 PecF (FW209).

In another embodiment, a rHVT according to the invention comprises (i) a nucleotide sequence encoding a F protein of Newcastle Disease Virus, or an immunogenic fragment or variant thereof, inserted into the non-coding region between UL45 and UL46 under control of a Pec promoter; and (ii) a nucleotide sequence encoding an Hemagglutinin (HA) protein of a subtype H9 avian influenza virus, or an immunogenic fragment or variant thereof, inserted into the non-coding region between SORF3 and US2 under control of a Hcmv promoter. Preferably, such a rHVT is a bivalent construct HVT/45-46 PecF/87-88 Hcmv H9-CNn1 (FW252), as described in the experimental data.

Particularly preferred rHVTs of the invention are prepared using the FC126 or PB1 strains.

The recombinant HVT of the present invention may be propagated in cell cultures. In preferred embodiments, CEF, embryonated eggs, chicken kidney cells, and the like are used as the host cells for the propagation of recombinant HVT. Multivalent recombinant HVT of the present invention may be cultured in a culture medium such as Eagle's MEM, Leibowitz-L-15/McCoy 5A (1:1 mixture) culture medium at about 370° C. for 3 to 4 days. The infected cells thus obtained are suspended in a culture medium containing 10% dimethyl sulfoxide (DMSO) and stored frozen under liquid nitrogen. Advantageously, the recombinant HVT of the invention present a high level of stability.

They are genetically stable, which means they maintain the inserted genes even after 10 or more passages, preferably after 15 passages, more preferably after 20 passages in cells of avian species, preferably CEF cells. They also provide stable expression of antigens, which means they coexpress the antigens in cells of avian species, preferably CEF cells, even after 10 or more passages, preferably after 15 passages, even more preferably after 20 passages. In the context of the invention a “passage” or “cell passaging” means a culture of cells in suitable conditions for allowing their growth and keeping them alive until they are 90% to 100% confluent. The passaging step consists on transferring a small number of cells of the previous confluent culture into a new culture medium. An aliquot of the previous confluent culture, containing a few cells, may be diluted in a large volume of fresh medium.

The viruses may be collected or purified using conventional techniques. They may be stored in any suitable medium, frozen and/or lyophilized.

Nucleic Acids and Cells

A further object of the invention relates to any nucleic acid contained in a virus as defined above. The nucleic acids may be single- or double-stranded, DNA or RNA, or variants thereof.

The invention also relates to a vector (e.g., plasmid, cosmid, artificial chromosome, etc.) comprising a nucleic acid of the invention.

The invention also relates to a cell containing a recombinant HVT, nucleic acid or vector of the invention. The cells are typically eukaryotic cells, such as avian cells, or prokaryotic cells (if the vector is suitable for replication or maintenance in such cell type).

Vaccine Compositions

The invention also relates to compositions, such as vaccines, which comprise a multivalent recombinant HVT of the invention, a nucleic acid of the invention, or a cell of the invention.

Vaccines of the invention typically comprise an immunologically effective amount of a recombinant HVT as described above, in a pharmaceutically acceptable vehicle.

The compositions and vaccines according to the present invention typically comprise a suitable solvent or diluent or excipient, such as for example an aqueous buffer or a phosphate buffer. The compositions may also comprise additives, such as proteins or peptides derived from animals (e.g., hormones, cytokines, co-stimulatory factors), nucleic acids derived from viruses and other sources (e.g., double stranded RNA, CpG), and the like which are administered with the vaccine in an amount sufficient to enhance the immune response. In addition, any number of combinations of the aforementioned substances may provide an immunopotentiation effect, and therefore, can form an immunopotentiator of the present invention.

The vaccines of the present invention may further be formulated with one or more further additives to maintain isotonicity, physiological pH and stability, for example, a buffer such as physiological saline (0.85%), phosphate-buffered saline (PBS), citrate buffers, Tris(hydroxymethyl aminomethane (TRIS), Tris-buffered saline and the like, or an antibiotic, for example, neomycin or streptomycin, etc.

The route of administration can be any route including oral, ocular (e.g., by eyedrop), oculo-nasal administration using aerosol, intranasal, Cloacal in feed, in water, or by spray, in ovo, topically, or by injection (e.g., intravenous, subcutaneous, intramuscular, intraorbital, intraocular, intradermal, and/or intraperitoneal) vaccination. The skilled person will easily adapt the formulation of the vaccine composition for each type of route of administration.

Each vaccine dose may contain a suitable dose sufficient to elicit a protective immune response in avian species. Optimization of such dose is well known in the art. The amount of antigen per dose may be determined by known methods using antigen/anti-body reactions, for example by the ELISA method.

The vaccines of the invention can be administered as single doses or in repeated doses, depending on the vaccination protocol.

The vaccines of the present invention are further advantageous in that they confer to bird species up to 100% protection against the targeted avian pathogens after 3 weeks of vaccination.

The present invention further relates to the use of the vaccine as described above for immunizing avian species, such as poultry, against a pathogen.

The present invention further relates to a method of immunizing avian species by administering an immunologically effective amount of the vaccine according to the invention. The vaccine may be advantageously administered intradermally, subcutaneously, intramuscularly, orally, in ovo, by mucosal administration or via oculo-nasal administration.

The present invention further relates to vaccination kits for immunizing avian species which comprises an effective amount of the multivalent vaccine as described above and a means for administering said components to said species. For example, such kit comprises an injection device filled with the multivalent vaccine according to the invention and instructions for intradermic, subcutaneous, intramuscular, or in ovo injection. Alternatively, the kit comprises a spray/aerosol or eye drop device filled with the multivalent vaccine according to the invention and instructions for oculo-nasal administration, oral or mucosal administration.

Further aspects and advantages of the present application will now be disclosed in the following examples, which are illustrative of the invention.

### EXAMPLES

In the following examples, recombinant HVT (rHVT) have been prepared and used. They are designated according to the following nomenclature:
HVT/insertion site, promoter, inserted antigen (monovalent construct);
HVT/first insertion site, first promoter, first inserted antigen/second insertion site, second promoter, second inserted antigen (bivalent construct).

List of rHVT prepared and used in the examples:
FW168: HVT/45-46 PecF (monovalent)
FW202: HVT/45-46 Mcmv ie1 H9-CN (monovalent)
FW204: HVT/45-46 Mcmv ie1 H9-CS (monovalent)
FW205: HVT/45-46 PecF/87-88 Mcmv ie1 H9-CS (bivalent)
FW206: HVT/45-46 PecF/87-88 Mcmv ie1 H9-CN (bivalent)
FW208: HVT/45-46 Mcmv ie1 H9-CS/87-88 PecF (bivalent)
FW209: HVT/45-46 Mcmv ie1 H9-CN/87-88 PecF (bivalent)
FW247: HVT/45-46 PecF/87-88 Mcmv ie1 H9-CNn1 (bivalent)
FW248: HVT/45-46 PecF/87-88 Mcmv ie1 H9-CNn2 (bivalent)
FW249: HVT/45-46 PecF/87-88 Mcmv ie1 H9-CNn3 (bivalent)
FW250: HVT/45-46 PecF/87-88 Mcmv ie1 H9-CNn4 (bivalent)
FW251: HVT/45-46 PecF/87-88 Mcmv ie1 H9-CNn5 (bivalent)
FW252: HVT/45-46 PecF/87-88 Hcmv H9-CNn1 (bivalent)

### Example 1: Design and Synthesis of Consensus Sequences of the Hemagglutinin Gene of Avian Influenza Virus H9 Subtype

Design and Synthesis of Consensus Sequence Called H9-CS

A consensus sequence of the hemagglutinin (HA) gene of avian influenza virus (AIV) H9 subtype was designed based on phylogenetic analyses and synthesized as follows, to maximize breadth of protection among H9N2 AIV isolates.

First, more than 80 HA gene sequences from recent H9N2 strains were collected from the public database. Then, a phylogenetic tree was constructed, and the center of tree (COT) sequence and the most recent common ancestor (MRCA) sequence were identified. The COT analysis and the MRCA analysis are computational methods that were developed to minimize negative impacts of antigenic diversity on vaccine immunogenicity (Kesturu et al., 2006). The inventors also looked into alignment of the sequences closely especially at predicted antigenic regions and N-glycosylation sites, in order to ensure selection of a representative sequence. These analyses led the inventors to the design of one sequence, called H9-CS, which provides broad cross-reactivity to various H9 strains.

The H9-CS gene sequence was synthesized artificially. The amino and nucleic acid sequences of H9-CS are provided as SEQ ID NO: 2 and SEQ ID NO: 8, respectively.

Design and Synthesis of Sequences H9-CNn1 to H9-CNn5

Five AIV H9 subtype antigens were designed and synthesized as follows, to maximize breadth of protection among H9N2 AIV isolates. The antigens were more particularly designed based on a known HA gene sequence from AIV H9 subtype, herein named H9-CN. (A/chicken/Henan/H24/2011; GenBank Acc. No. JN804297).

Several sequences of H9 strains were collected, including seven Chinese strains isolates of 2016. Computational protein modeling analysis was conducted based on those H9 sequences and led the inventors to a selection of four antigens called H9-CNn1-4 (SEQ ID NO: 4), H9-CNn2 (SEQ ID NO: 5), H9-CNn3 (SEQ ID NO: 6), H9-CNn4 (SEQ ID NO: 7). H9-CNn5 was designed by replacing the Transmembrane domain (TM) of H9-CNn1 with that of H3 subtype of AIV, in order to further increase molecular stability and cross reactivity.

The amino acid sequences of H9-CNn1-5 are provided as SEQ ID NOs: 3-7, respectively. Nucleic acid sequences encoding H9-CNn1-5 are provided as SEQ ID NOs: 9-13, respectively

### Example 2: Construction of Recombinant HVT

2.1. Construction of Homology Vectors

The plasmid construction was essentially performed by the standard molecular biology techniques (Molecular Cloning: A Laboratory Manual. 4th Edition, Cold Spring Harbor Laboratory, Cold Spring Harbor, New York, USA, 2012).

Construction Of p45/46Mcmv Ie1 H9-CS and p45/46Mcmv Ie1 H9-CN

The murine cytomegalovirus (Mcmv) ie1 promoter (SEQ ID NO: 16) was synthesized in pUC18 based vector, resulting in pGI Mcmv ie1.

A polyA signal (SPA: SEQ ID NO:17) was also synthesized and inserted into pGI Mcmv ie1 cleaved with SalI and SfiI, resulting in pGI Mcmv ie1 SPA. The Mcmv ie1 promoter—SPA cassette was cut out from pGI Mcmv ie1 SPA by BglI digestion and inserted into SfiI site of p45/46Sfi (WO03/064595), resulting in p45/46 Mcmv ie1 SPA.

The consensus sequence H9-CS (SEQ ID NO:8) synthesized in example 1 was used. The gene sequence H9-CN (A/chicken/Henan/H24/2011; GenBank Acc. No. JN804297) referred to in example 1 was synthesized and used. These HA sequences were digested with XbaI and SalI, and then inserted into XbaI & SalI-cleaved p45/46 Mcmv ie1 SPA, resulting in p45/46 Mcmv ie1 H9-CS SPA and p45/46 Mcmv ie1 H9-CN SPA, respectively.

Construction of pHVT87-88 Mcmv ie1 H9-CS and pHVT87-88 Mcmv ie1 H9-CN

The Mcmv ie1 promoter—SPA cassette cut out from pGI Mcmv ie1 SPA by BglI digestion and inserted into SfiI site of pHVT87-88 (WO2013/144355), resulting in pHVT87-88 Mcmv ie1 SPA. Then, the H9-CS gene or the H9-CN gene digested with XbaI and SalI was inserted into XbaI & SalI-cleaved pHVT87-88 Mcmv ie1 SPA, resulting in pHVT87-88 Mcmv ie1 H9-CS SPA and pHVT87-88 Mcmv ie1 H9-CN SPA, respectively.

Construction of pHVT87-88 PecF

The Pec promoter—Newcastle disease virus (NDV) F gene—SV40 polyA cassette was taken from p45/46PecF (WO03/064595) by BglI digestion and cloned into SfiI-digested pHVT87-88, resulting in pHVT87-88 PecF. NDV F gene used comprises SEQ ID NO: 14.

Construction of pHVT87-88 Mcmv Ie1 H9-CNn1 to CNn5

The H9-CNn1 (SEQ ID NO: 9), H9-CNn2 (SEQ ID NO: 10), H9-CNn3 (SEQ ID NO: 11), H9-CNn4 (SEQ ID NO: 12), and H9-CNn5 (SEQ ID NO: 13) genes synthesized in example 1 were used. These genes were cloned into XbaI & SalI-cleaved pHVT87-88 Mcmv ie1 SPA, resulting in pHVT87-88 Mcmv ie1 H9-CNn1 SPA, pHVT87-88 Mcmv ie1 H9-CNn2 SPA, pHVT87-88 Mcmv ie1 H9-CNn3 SPA, pHVT87-88 Mcmv ie1 H9-CNn4 SPA, and pHVT87-88 Mcmv ie1 H9-CNn5 SPA.

Construction of pHVT87-88 cmv H9-CNn1

Human cytomegalovirus (Hcmv) promoter was taken from pGICMVpA (WO2008/121329) by BglI and XbaI digestion and inserted into BglI & XbaI-cleaved pHVT87-88 Mcmv ie1 H9-CNn1 SPA, resulting in pHVT87-88 Hcmv H9-CNn1 SPA.

2.2 Construction of Recombinant HVT

Construction of recombinant HVT (rHVT) was conducted by homologous recombination in cultured cells. HVT DNA was prepared from chicken embryo fibroblasts (CEF) infected with parent HVT, as described by Morgan et al. (Avian Diseases, 34:345-351, 1990). Approximately 2 μg of the HVT DNA and 1 μg of one of the homology vectors were transfected into approximately 10⁷ CEF cells by electroporation using Nucleofector II (Lonza, Basel, Switzerland). The transfected cells were added to Leibovitz's L-15 (Life Technologies Corp., Cat. #41300-39), McCoy's 5A Medium (Life Technologies Corp., Cat. #21500-061) (1:1) and 4% calf serum (LM (+) medium), planted in 96-well tissue culture plates, and incubated at 37° C. in 4-5% CO₂ for 5-7 days until HVT plaques became visible. The cells were then detached from the plates by trypsinization, transferred equally to two 96-well plates with CEF and incubated for 3 to 5 days until plaques were observed. Screening was conducted by the black plaque assay, staining only plaques expressing antigen proteins, NDV F protein or AIV HA protein. Briefly, one of the two plates was fixed with methanol:acetone mixture (1:2) and incubated with rabbit anti-NDV F protein sera or chicken anti-HA (H9) sera. The plate was then incubated with biotinylated anti-rabbit IgG antibody (Vector Laboratories, Cat #BA-1000) or biotinylated anti-chicken IgY antibody (Vector Laboratories, Cat #BA-9010), and finally with VECTASTAIN ABC-AP kit (Vector Laboratories, Cat #AK-5000). Plaques expressing the antigens were stained by addition of NBT/BCIP solution (Roche Applied Science, Cat #1681451). Wells containing stained recombinant plaques were identified and cells in the corresponding wells on the other 96-well plate were trypsinized. The cells were then diluted in fresh secondary CEF cells and transferred to new 96-well plates to complete the first round of purification. The purification procedure was repeated until all plaques were stained positively in the black plaque assay. Multiple clones were isolated for each construct.

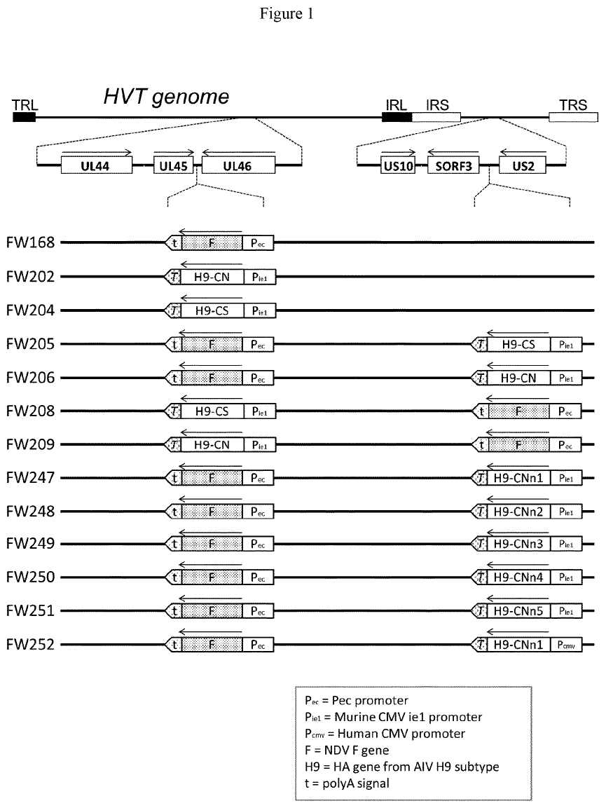

A list of the constructed rHVT, their parent viruses and the homology vectors used is provided in Table 1 below. A diagram showing genomic structures of the rHVT is provided in FIG. 1. For construction of bivalent rHVT expressing both the NDV F gene and the AIV HA-H9 gene (rHVT/ND-H9), the aforementioned construction process was repeated using viral DNA extracted from rHVT containing one antigen gene.

TABLE 1
constructed rHVT, parent viruses, and homology vectors

| Construct code | Parent virus | Homology vector |
|---|---|---|
| FW168 | HVT FC-126 | p45/46PecF |
| (monovalent) | strain | |
| FW202 | HVT FC-126 | p45/46 Mcmv ie1 H9-CN SPA |
| (monovalent) | strain | |
| FW204 | HVT FC-126 | p45/46 Mcmv ie1 H9-CS SPA |
| (monovalent) | strain | |
| FW205 (bivalent) | FW168 | pHVT87-88 Mcmv ie1 H9-CS SPA |
| FW206 (bivalent) | FW168 | pHVT87-88 Mcmv ie1 H9-CN SPA |
| FW208 (bivalent) | FW204 | pHVT87-88 PecF |
| FW209 (bivalent) | FW202 | pHVT87-88 PecF |
| FW247 (bivalent) | FW168 | pHVT87-88 Mcmv ie1 H9-CNn1 SPA |
| FW248 (bivalent) | FW168 | pHVT87-88 Mcmv ie1 H9-CNn2 SPA |
| FW249 (bivalent) | FW168 | pHVT87-88 Mcmv ie1 H9-CNn3 SPA |
| FW250 (bivalent) | FW168 | pHVT87-88 Mcmv ie1 H9-CNn4 SPA |
| FW251 (bivalent) | FW168 | pHVT87-88 Mcmv ie1 H9-CNn5 SPA |
| FW252 (bivalent) | FW168 | pHVT87-88 Hcmv H9-CNn1 SPA |

### Example 3: Expression of Inserted Antigens by Recombinant HVT

Expression of the NDV F protein and/or the AIV HA-H9 protein by the rHVT constructs prepared in Example 2 was confirmed by the immunofluorescence assay (IFA) and the Western blot assay. For the IFA, CEF monolayer with rHVT plaques was fixed with methanol:acetone mixture (1:2) and incubated with a mixture of rabbit anti-NDV F protein sera and chicken anti-HA (H9) sera. The plate was then incubated with a mixture of Alexa Fluor 488 anti-rabbit IgG antibody (Invitrogen, Cat #A-11008) and Alexa Fluor 546 anti-chicken IgY antibody (Invitrogen, Cat #A-11040), and observed under a fluorescence microscope. Specific green (F protein) or red (HA-H9 protein) fluorescence was observed with each rHVT, demonstrating that these rHVT express the antigen proteins. Also, it was demonstrated that each plaque of bivalent rHVT/ND-H9 constructs express both F and HA-H9 antigens (FIG. 2).

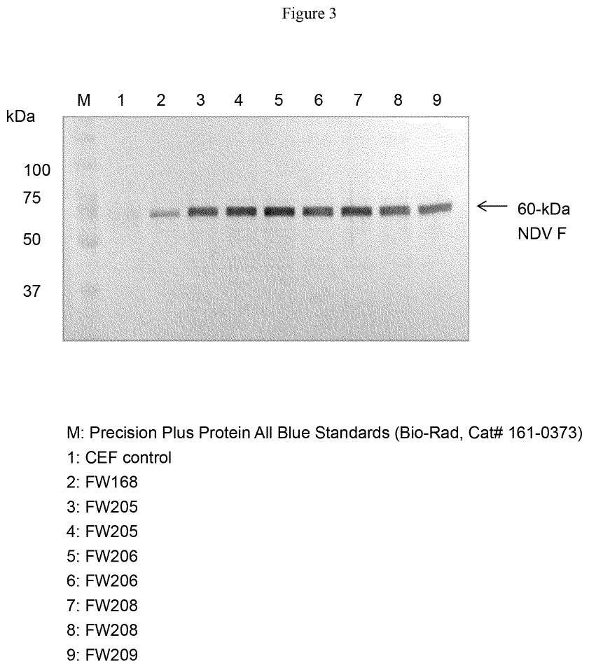

The western blot was conducted using CEF cells infected with the recombinant viruses and rabbit anti-NDV F protein sera or chicken anti-HA (H9) sera. Briefly, CEF cells in 6-well plates were infected with one of the recombinant viruses or the parent HVT strain at a multiplicity of infection of approximately 0.1. Three days post inoculation, cells were harvested with trypsin and centrifuged at 913×g for 5 minutes. The pellet was washed with PBS and resuspended with 100 μl of PBS. After adding the same volume of 2×SDS sample buffer (130 mM Tris-Cl (pH6.8), 6% SDS, 20% Glycerol, 10% 2-Mercaptoethanol and 0.01% Bromo Phenol Blue), cell suspension was boiled for 5 minutes. The samples were separated by SDS-PAGE using 10% polyacrylamide gel and transferred to a PVDF membrane (Immobilon-P, Millipore). The membrane was dried completely and then incubated with rabbit anti-NDV F protein sera or chicken anti-HA (H9) sera. After the antibody was washed off, the membrane was incubated with either biotinylated anti-rabbit IgG antibody (Vector Laboratories, Cat #BA-1000) or biotinylated anti-chicken IgY antibody (Vector Laboratories, Cat #BA-9010) and then with VECTASTAIN ABC-AP kit (Vector Laboratories, Cat #AK-5000). Protein bound with the antibody was visualized by adding NBT/BCIP solution. As shown in FIGS. 3 to 6, a protein band of 60 kilodaltons (kDa) for NDV F protein or a protein band of 70 kDa for AIV HA-H9 protein was observed only in the lane with the recombinant virus infected cells, confirming that the rHVT constructs prepared in Example 2 express the antigen proteins of the expected sizes.

### Example 4: Verification of Genome Structure of Recombinant HVT

Genome structures of the rHVT constructs prepared in Example 2 were verified by two PCR reactions amplifying the two inserted regions (UL45/UL46 and SORF3/US2). The primer pairs used in the PCR reactions are SEQ ID NO: 18 (5′-GGGGAAGTCTTCCGGTTAAGGGAC-3′) and SEQ ID NO: 19 (5′-GGTGCAATTCGTAAGACCGATGGG-3′) for UL45/UL46, and SEQ ID NO: 20 (5′-GCGCGACTCCATACATTGA-3′) and SEQ ID NO: 21 (5′−AGTCCACATGCACCCC ACCTAAAC-3′) for (SORF3/US2). Expected sizes of PCR products containing the inserted genes were observed with all of the rHVT, confirming that these recombinant HVT have the expected genome structures.

### Example 5: Genetic Stability of Recombinant HVT

The rHVT constructs prepared in example 2 were passed in CEF 20 times and tested for genetic stability. All of the rHVT after 20×passages were tested by PCR as described in Example 4 for genome structure and by IFA and the western blot for expression of the antigen proteins as described in Example 3. It was shown that all of the rHVT constructs maintained the inserted genes by PCR and expressed the antigens proteins by IFA and the western blot, demonstrating that these rHVT are genetically stable and provide stable expression of antigens.

### Example 6: Antibody Titers in Chickens Vaccinated with FW205, FW206, or FW208

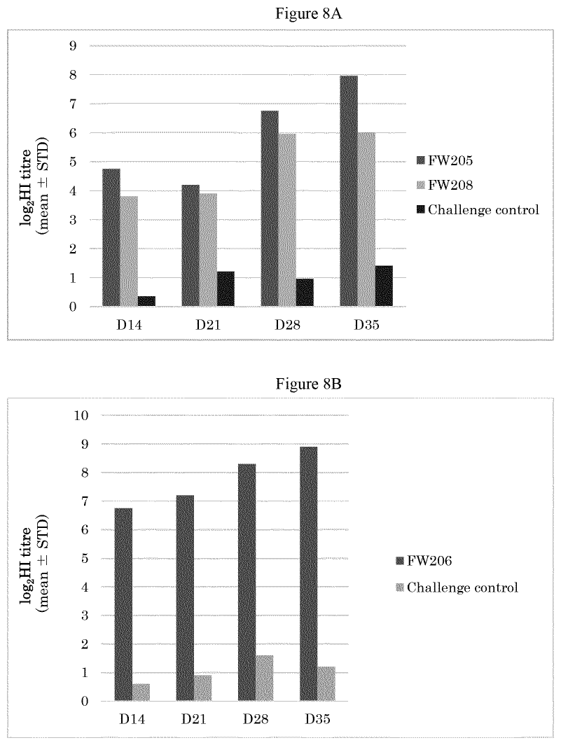

Constructs FW205, FW206, and FW208 were investigated for their ability to induce antibodies against NDV F and AIV HA-H9. Approximately 1,000 plaque forming units (PFU) of rHVT construct were administered subcutaneously to specific pathogen free (SPF) chickens at one day of age. Sera were collected each week between 2 to 5 weeks of age and tested for specific antibodies for the antigens. Antibodies against NDV F were tested using ID Screen Newcastle Disease Indirect ELISA kit (ID Vet). All of the constructs tested induced antibodies against NDV F protein (FIG. 7). FW205 induced exceptionally higher titers than the other constructs. Antibodies against AIV HA-H9 were tested by hemagglutin inhibition (HI) tests as described in Chapter 3.3.4 (Avian Influenza) of OIE Terrestrial Manual 2018, using inactivated AIV of H9 subtype. All of the tested constructs were shown to exhibit HI titers between 2 and 5 weeks of age (FIGS. 8A and 8B).

### Example 7: Protection Against ND in Chickens Vaccinated with FW205, FW206, or FW208, after Challenge at 17 Days of Age

Efficacy of constructs FW205 (HVT/45-46 PecF/87-88 Mcmv ie1 H9-CS), FW206 (HVT/45-46 PecF/87-88 Mcmv ie1 H9-CN) and FW208 (HVT/45-46 Mcmv ie1 H9-CS/87-88 PecF) was investigated against challenge with virulent NDV strain. SPF chickens at one day of age were vaccinated subcutaneously with approximately 1,000 PFU of one of the rHVT/ND-H9 constructs. The chickens at 17 days of age were challenged with 10⁵ ELD₅₀ of virulent NDV Herts 33/56 strain via intramuscular injection and observed 14 days for clinical signs of Newcastle disease (ND). All constructs provided protection of 70% or higher against the challenge at the very early age.

TABLE 2
Protection against ND in SPF chickens vaccinated with FW205,
FW206 and FW208, after challenge at 17 days of age

| Group | | # | # with ND | |
|---|---|---|---|---|
| number | Group | chickens | clinical signs | % protection |
| 1 | FW205 | 15 | 3 | 80% |
| 3 | FW206 | 15 | 2 | 87% |
| 5 | FW208 | 15 | 4 | 73% |
| 9 | NICC | 10 | 10 | 0% |

NICC = non-immunized, challenged positive controls

### Example 8: Protection Against ND in Chickens Vaccinated with FW205, FW206, or FW208, after Challenge at 21 Days of Age

Efficacy of FW205, FW206, and FW208 was investigated against challenge with virulent NDV strain. FW168 (HVT/45-46 PecF) was also tested. SPF chickens at one day of age were vaccinated subcutaneously with approximately 400 PFU of one of rHVT constructs. The chickens at 21 days of age were challenged with 10⁵ ELD₅₀ of virulent NDV Herts 33/56 strain via intramuscular injection and observed 14 days for clinical signs of Newcastle disease (ND). Constructs FW205 and FW168 provided excellent protection, 100% and 96%, respectively, and the other constructs also provided good protection above 70%. Results are shown in Table 3 below.

TABLE 3
Protection against ND in SPF chickens vaccinated with FW205,
FW206, FW208, and FW168, after challenge at 21 days of age

| Group | | # | # with ND | |
|---|---|---|---|---|
| number | Group | chickens | clinical signs | % protection |
| 1 | FW205 | 24 | 0 | 100% |
| 3 | FW206 | 24 | 7 | 71% |
| 4 | FW208 | 24 | 5 | 79% |
| 5 | FW168 | 24 | 1 | 96% |
| 6 | NICC | 12 | 12 | 0% |

NICC = non-immunized, challenged positive controls

### Example 9: Protection Against AI in Chickens Vaccinated with FW205, FW206, or FW208, after Challenge at 25 Days of Age

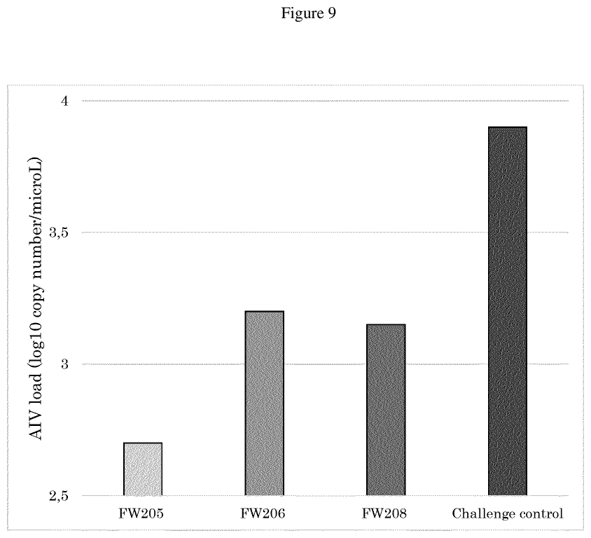

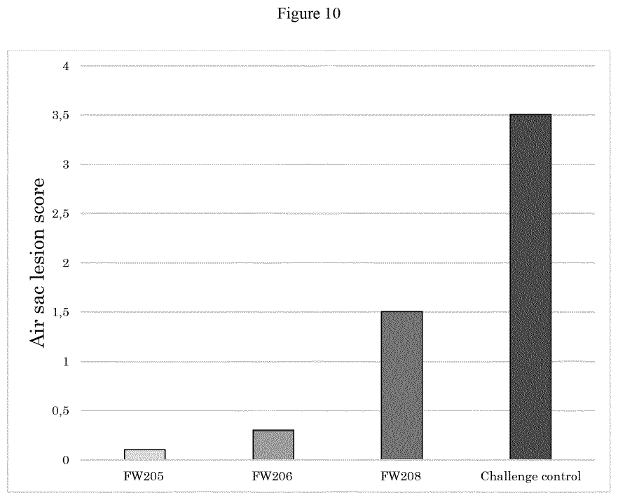

Efficacy of FW205, FW206 and FW208 against challenge with AIV H9 subtype was investigated in commercial broiler chickens. Commercial broiler chickens at one day of age were vaccinated subcutaneously with approximately 1,000 PFU of FW205, FW206, or FW208. The chickens were challenged with 10⁷ EID₅₀ of AIV A/chicken/Saudi Arabia/D1816/1/1/2011 (H9N2) strain via intratracheal and intranasal route at 25 days of age. Tracheal samples were collected at 5 days post challenge and used for quantification of AIV by qPCR analysis. At 11 days post challenge, the chickens were necropsied and lesions in air sacs were evaluated. As shown FIG. 9, all vaccinated groups had lower viral load as compared to the control group. FW205 reduced AIV load in tracheal swabs by 1 log compared to the challenge control group (FIG. 9), and AIV loads in the other two groups vaccinated either with FW206 or FW208, were 0.5 log lower than that in the challenge control (FIG. 9). Air sac lesion scores in all vaccinated groups were also substantially lower than in challenge control group (FIG. 10). These results demonstrated that all tested constructs, provided clinical protection against challenge with AIV H9 subtype.

### Example 10: Antibody Titers in Chickens Vaccinated with FW247, FW248, FW249, FW250, FW251 or FW252

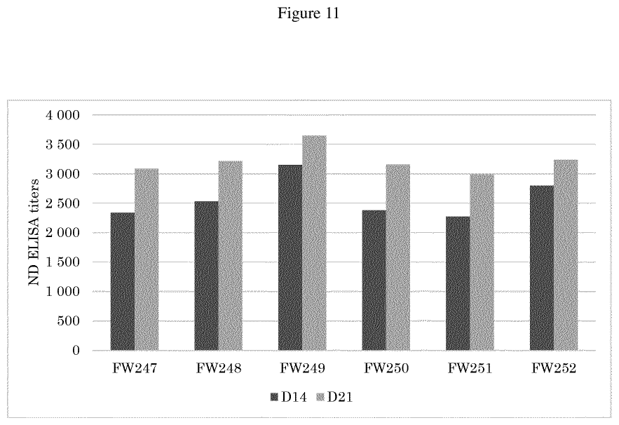

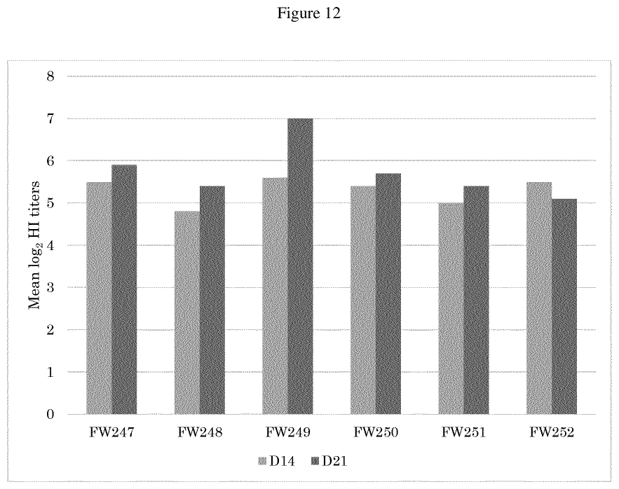

Constructs FW247, FW248, FW249, FW250, FW251, and FW252 were investigated for their ability to induce antibodies against NDV F and AIV HA-H9. Approximately 3,000 PFU of rHVT construct were administered subcutaneously to SPF chickens at one day of age. Sera were collected at 2 and 3 weeks of age and tested for specific antibodies for the antigens. Antibodies against NDV F were tested using ID Screen Newcastle Disease Indirect ELISA kit (ID Vet). Antibodies against AIV HA-H9 were tested by the HI tests using inactivated AIV of H9 subtype. All of the constructs tested induced antibodies against both NDV F protein (FIG. 11) and AIV HA-H9 (FIG. 12). Construct FW249 appeared to induce high F antibodies and HA-H9 antibodies.

### Example 11: Protection Against ND in Chickens Vaccinated with FW249, after Challenge at 21 Days of Age

Efficacy of construct FW249 was investigated against challenge with virulent NDV strain. SPF chickens at one day of age were vaccinated subcutaneously with approximately 2,500 PFU of one of the rHVT/ND-H9 construct. The chickens at 21 days of age were challenged with 10⁵ ELD₅₀ of virulent NDV Herts 33/56 strain via intramuscular injection and observed 14 days for clinical signs of Newcastle disease (ND). Results are provided in Table 4.

TABLE 4
Protection against ND in SPF chickens vaccinated
with FW249, after challenge at 21 days of age

| Group | | # | # with ND | |
|---|---|---|---|---|
| number | Group | chickens | clinical signs | % protection |
| 1 | FW249 | 13 | 1 | 92% |
| 2 | NICC | 13 | 13 | 0% |

NICC = non-immunized, challenged positive controls

SEQUENCE LISTING

SEQ ID NO: 1
Name: F protein
Type: amino acid
Length: 553
MGSRSSTRIPVPLMLTVRIMLALSCVCPTSSLDGRPLAAAGIVVTGDKAVNIYTSSQTGSIIIKLL
PNMPKDKEACAKAPLEAYNRTLTTLLTPLGDSIRRIQESVTTSGGGKQGRLIGAIIGGVALGVATA
AQITAASALIQANONAANILRLKESIAATNEAVHEVTDGLSQLAVAVGKMQQFVNDQFNKTAQELD
CIKITQQVGVELNLYLTELTTVFGPQITSPALTQLTIQALYNLAGGNMDYLLTKLGVGNNQLSSLI
GSGLITGNPILYDSQTQLLGIQVTLPSVGNLNNMRATYLETLSVSTTKGFASALVPKVVTQVGSVI
EELDTSYCIETDLDLYCTRIVTFPMSPGIYSCLSGNTSACMYSKTEGALTTPYMTLKGSVIANCKM
TTCRCADPPGIISQNYGEAVSLIDROSCNILSLDGITLRLSGEFDATYQKNISIQDSQVIVTGNLD
ISTELGNVNNSISNALDKLEESNSKLDKVNVKLISTSALITYIVLIVISLVCGILSLVLACYLMYK
QKAQQKTLLWLGNNTLDQMRATTKM
SEQ ID NO: 2
Name: (H9-CS)
Type: amino acid
Length: 560
METISLMTILLVVTTSNADKICIGHQSTNSTETVDTLTETNVPVTHAKELLHTEHNGMLCATNLGH
PLILDTCTIEGLVYGNPSCDLLLGGREWSYIVERPSAVNGTCYPGNVENLEELRTLFSSSSSYQRI
QIFPDTIWNVTYTGTSKSCSDSFYRNMRWLTQKNGVYPVQDAQYTNNRGKDILFVWGIHHPPTDTA
GHVLSGESHGRILKTDLNSGNCVVQCQTEKGGLNSTLPFHNISKYAFGTCPKYIGVKSLKLAIGLR
NVPARSSRGLFGAIAGFIEGGWPGLVAGWYGFQHSNDQGVGMAADRDSTQKAVDKITSKVNNIVDK
MNKQYEIIDHEFSEVETRLNMINNKIDDQIQDVWAYNAELLVLLENQKTLDEHDANVNNLYNKVKR
ALGSNAIEDGKGCFELYHKCDNQCMETIRNGTYNRRKYKEESRLERQKIEGVKLESEGTYKILTIY
STVASSLVLAMVFAAFLFWAMSNGSCRCNICI
SEQ ID NO: 3
Name: H9-CNn1
Type: amino acid
Length: 560
MEVVSLITILLVVTVSNADKICIGYQSTNSTETVDTLTENNVPVTHAKELLHTEHNGMLCATSLGQ
PLILDTCTIEGLIYGNPSCDLSLEGREWSYIVERPSAVNGLCYPGNVENLEELRSLFSSARSYQRI
QIFPDTIWNVSYDGTSTACSGSFYRSMRWLTRKNGDYPIQDAQYTNNQGKNILFMWGINHPPTDDT
QRNLYTRTDTTTSVATEEINRIFKPLIGPRPLVNGLMGRIDYYWSVLKPGQTLRIKSDGNLIAPWY
GHILSGESHGRILKTDLKRGSCTVQCQTEKGGLNTTLPFQNVSKYAFGNCSKYIGIKSLKLAVGLR
NVPSRSSRGLFGAIAGFIEGGWSGLVAGWYGFQHSNDQGVGMAADRDSTQKAIDKITSKVNNIVDK
MNKQYEIIDHEFSEVETRLNMINNKIDDQIQDIWAYNAELLVLLENQKTLDEHDANVNNLYNKVKR
ALGTNAVEDGKGCFELYHKCDDQCMETIRNGTYNRRKYQEESKLERQKIEGVKLESEGTYKILTIY
STVASSLVIAMGFAAFLFWAMSNGSCRCNICI
SEQ ID NO: 4
Name: H9-CNn2
Type: amino acid
Length: 560
MEVVSLITILLVVTVSNADKICIGYQSTNSTETVDTLTENNVPVTHAKELLHTEHNGMLCATSLGQ
PLILDTCTIEGLIYGNPSCDLSLEGREWSYIVERPSAVNGLCYPGNVENLEELRSLFSSARSYQRI
QIFPDTIWNVSYDGTSTACSGSFYRSMRWLTRKNGDYPTQDAQYTNNQGKNILFMWGINHPPTDTA
QTNLYTRTDTTTSVATEEINRIFKPLIGPRPLVNGLMGRIDYYWSVLKPGQTLRIKSDGNLIAPWY
GHILSGESHGRILKTDLKRGSCTVQCQTEKGGLNTTLPFQNVSKYAFGNCSKYIGIKSLKLAVGLR
NVPSRSSRGLFGAIAGFIEGGWSGLVAGWYGFQHSNDQGVGMAADRDSTQKAIDKITSKVNNIVDK
MNKQYEIIDHEFSEVETRLNMINNKIDDQIQDIWAYNAELLVLLENQKTLDEHDANVNNLYNKVKR
ALGTNAVEDGKGCFELYHKCDDQCMETIRNGTYNRRKYQEESKLERQKIEGVKLESEGTYKILTIY
STVASSLVIAMGFAAFLFWAMSNGSCRCNICI
SEQ ID NO: 5
Name: H9-CNn3
Type: amino acid
Length: 560
MEVVSLITILLVVTVSNADKICIGYQSTNSTETVDTLTENNVPVTHAKELLHTEHNGMLCATSLGQ
PLILDTCTIEGLIYGNPSCDLSLEGREWSYIVERPSAVNGLCYPGNVENLEELRSLFSSARSYQRI
QIFPDTIWNVSYDGTSTACSGSFYRSMRWLTQKNNAYPIQDAQYTNNQGKNILFMWGINHPPTDTT
QRNLYTRTDTTTSVATEEINRIFKPLIGPRPLVNGLMGRIDYYWSVLKPGQTLRIKSDGNLIAPWY
GHILSGESHGRILKTDLKRGSCTVQCQTEKGGLNTTLPFQNVSKYAFGNCSKYIGIKSLKLAVGLR
NVPSRSSRGLFGAIAGFIEGGWSGLVAGWYGFQHSNDQGVGMAADRDSTQKAIDKITSKVNNIVDK
MNKQYEIIDHEFSEVETRLNMINNKIDDQIQDIWAYNAELLVLLENQKTLDEHDANVNNLYNKVKR
ALGTNAVEDGKGCFELYHKCDDQCMETIRNGTYNRRKYQEESKLERQKIEGVKLESEGTYKILTIY
STVASSLVIAMGFAAFLFWAMSNGSCRCNICI
SEQ ID NO: 6
Name: H9-CNn4
Type: amino acid
Length: 560
MEVVSLITILLVVTVSNADKICIGYQSTNSTETVDTLTENNVPVTHAKELLHTEHNGMLCATSLGQ
PLILDTCTIEGLIYGNPSCDLLLGGREWSYIVERPSAVNGLCYPGNVENLEELRSLFSSARSYQRI
QIFPDTIWNVSYDGTSTACSGSFYRSMRWLTRKNGDYPIQDAQYTNNQGKNILFMWGINHPPTDDT
QRNLYTRTDTTTSVATEEINRIFKPLIGPRPLVNGLMGRIDYYWSVLKPGQTLRIKSDGNLIAPWY
GHILSGESHGRILKTDLKRGSCTVQCQTEKGGLNTTLPFQNVSKYAFGNCSKYIGIKSLKLAVGLR
NVPSRSSRGLFGAIAGFIEGGWSGLVAGWYGFQHSNDQGVGMAADRDSTQKAIDKITSKVNNIVDK
MNKQYEIIDHEFSEVETRLNMINNKIDDQIQDIWAYNAELLVLLENQKTLDEHDANVNNLYNKVKR
ALGTNAVEDGKGCFELYHKCDDQCMETIRNGTYNRRKYQEESKLERQKIEGVKLESEGTYKILTIY
STVASSLVIAMGFAAFLFWAMSNGSCRCNICI
SEQ ID NO: 7
Name: H9-CNn5
Type: amino acid
Length: 559
MEVVSLITILLVVTVSNADKICIGYQSTNSTETVDTLTENNVPVTHAKELLHTEHNGMLCATSLGQ
PLILDTCTIEGLIYGNPSCDLSLEGREWSYIVERPSAVNGLCYPGNVENLEELRSLFSSARSYQRI
QIFPDTIWNVSYDGTSTACSGSFYRSMRWLTRKNGDYPIQDAQYTNNQGKNILFMWGINHPPTDDT
QRNLYTRTDTTTSVATEEINRIFKPLIGPRPLVNGLMGRIDYYWSVLKPGQTLRIKSDGNLIAPWY
GHILSGESHGRILKTDLKRGSCTVQCQTEKGGLNTTLPFQNVSKYAFGNCSKYIGIKSLKLAVGLR
NVPSRSSRGLFGAIAGFIEGGWSGLVAGWYGFQHSNDQGVGMAADRDSTQKAIDKITSKVNNIVDK
MNKQYEIIDHEFSEVETRLNMINNKIDDQIQDIWAYNAELLVLLENQKTLDEHDANVNNLYNKVKR
ALGTNAVEDGKGCFELYHKCDDQCMETIRNGTYNRRKYQEESKLERQKIEGVKLESEGTYKILTIS
FAISCFLLCVVLLGFIMWACQNGSCRCNICI
SEQ ID NO: 8
Name: H9-CS
Type: nucleotide
Length: 1683
atggaaacaatatcactgatgactatactactagtagtaacaacaagcaatgcagataaaatctgc
attggccaccagtcaacaaattccacagaaactgtggacacgctaacagaaactaatgttcctgtg
acacatgccaaagaattgctccacacagagcacaatggaatgctgtgtgcaacaaatctgggacat
cccctcatcctagacacatgcactatcgaaggacttgtctatggtaacccttcttgtgatctgttg
ttggggggaagagaatggtcctacatcgttgaaagaccatcagctgtaaatggaacgtgttaccct
gggaatgtggaaaacttagaggaactcagaacactctttagttcctctagttcctaccaaagaatc
caaatattcccagacacaatctggaatgtgacttacactggaacaagcaaatcatgttcagattca
ttctacaggaatatgagatggctgactcaaaagaacggggtttatcctgttcaagacgcccaatac
acaaataatcggggaaaagacattcttttcgtgtggggtatacatcatccacccaccgatactgca
cagacgaatttgtacacaagaaccgacacaacaacaagtgtaacaacagaaaatttagatagaacc
ttcaaaccattgatagggccaaggccccttgtcaatggtctgattggaagaattaattattattgg
tcagtactaaaaccaggccagacattgcgagtaagatccaatgggaatttgattgctccatggttt
ggacacgttctctcaggggagagccatggtagaatcctgaaaactgatttaaacagtggtaattgt
gtagtgcaatgtcagactgaaaaaggtggcctaaacagtacattgcctttccacaatatcagtaaa
tatgcatttgggacttgccccaaatatattggagtcaagagtctcaaactggcaatcggtctgaga
aacgtgcctgctaggtcaagtagaggactatttggagccatagctggattcatagaaggaggttgg
ccagggctagttgccggttggtatggtttccagcattcaaatgatcaaggggttggtatggcagca
gatagggattcaactcaaaaggcagttgataaaataacatccaaggtgaataatatagtggacaag
atgaacaagcaatatgaaataattgatcatgaattcagtgaggttgaaactagactcaatatgatc
aataataagattgatgaccaaatacaagatgtatgggcatataatgcagagttgctagtactactt
gagaaccagaaaacactcgatgagcatgacgcaaacgtgaacaacctatataacaaggtgaaaagg
gccttgggctccaatgcgattgaagatgggaagggctgtttcgagctataccacaaatgtgataac
caatgcatggaaactattcggaatgggacctataataggaggaagtacaaagaggaatcaagacta
gaaagacagaaaatagagggggtcaaactggaatctgaggggacttacaaaatacttaccatttat
tcgactgtcgcctcatctcttgtacttgcaatggtgtttgctgccttcttattctgggccatgtcc
aatggatcatgcaggtgcaacatctgtatataa
SEQ ID NO: 9
Name: H9-CNn1
Type: nucleotide
Length: 1683
atggaagtagtatcactaataactatactactagtagtaacagtaagcaatgcagataaaatctgc
atcggctaccaatcaacaaactccacagaaactgtggacacactaacagaaaacaatgtccctgtg
acacatgccaaagaactgctccacacagagcataatgggatgctgtgtgcaacaagcttgggacaa
cctcttattttagacacctgcaccattgaagggctaatctatggcaatccttcttgtgatctatcg
ctggaaggaagagaatggtcctatatcgtcgagagaccatcagctgttaacggattgtgttacccc
gggaatgtagaaaacctagaagagctaaggtcactttttagttctgctaggtcttatcaaagaatc
cagattttcccagacacaatctggaatgtgtcttacgatggaacaagcacagcatgctcaggttca
ttctacagaagcatgagatggttgactcgaaagaacggcgattaccctatccaagacgcccaatac
acaaataatcaagggaagaacattcttttcatgtggggcataaatcacccacccaccgatgatacg
cagagaaatctgtacacgagaaccgacacaacaacgagtgtggcaacagaagaaataaataggatc
ttcaaaccattgataggaccaaggcctcttgtcaacggtttgatgggaagaattgattattattgg
tctgtattgaaaccgggtcaaacactgcgaataaaatctgatgggaatctaatagctccatggtat
ggacacattctttcaggagagagccatggaagaatcctgaagactgatttaaaaaggggtagctgc
acagtgcaatgtcagacagagaaaggtggcttaaacacaacactgccattccaaaatgtaagtaag
tatgcatttggaaactgctcaaaatacattggcataaagagtctcaaacttgcagttggtctgagg
aatgtgccttccagatctagtagaggactattcggggccatagcaggattcatagagggaggttgg
tcagggctagttgctggttggtatggattccagcattcaaatgaccaaggggttggtatggcagca
gatagagactcaacccaaaaggcaattgataaaataacatccaaagtgaataacatagtggacaaa
atgaacaagcagtatgaaattattgatcatgaattcagtgaggttgaaactagacttaacatgatc
aataataagattgatgatcaaattcaagatatatgggcatataatgcagaattgctagttctgctt
gagaaccagaaaacactcgatgagcatgatgcaaatgtaaataatctatataataaagtgaagagg
gcattgggtaccaatgcggtggaagatgggaaaggatgtttcgagctataccacaaatgtgatgac
cagtgcatggagacaattcggaacgggacctacaacaggaggaagtatcaagaagaatcaaaatta
gaaaggcagaaaatagagggggtcaagctggaatctgaaggaacttacaaaatcctcaccatttat
tcgactgtcgcctcatctcttgtaattgcaatggggtttgctgccttcttgttctgggccatgtcc
aatgggtcttgcagatgcaacatttgtatataa
SEQ ID NO: 10
Name: H9-CNn2
Type: nucleotide
Length: 1683
atggaagtagtatcactaataactatactactagtagtaacagtaagcaatgcagataaaatctgc
atcggctaccaatcaacaaactccacagaaactgtggacacactaacagaaaacaatgtccctgtg
acacatgccaaagaactgctccacacagagcataatgggatgctgtgtgcaacaagcttgggacaa
cctcttattttagacacctgcaccattgaagggctaatctatggcaatccttcttgtgatctatcg
ctggaaggaagagaatggtcctatatcgtcgagagaccatcagctgttaacggattgtgttacccc
gggaatgtagaaaacctagaagagctaaggtcactttttagttctgctaggtcttatcaaagaatc
cagattttcccagacacaatctggaatgtgtcttacgatggaacaagcacagcatgctcaggttca
ttctacagaagcatgagatggttgactcgaaagaacggcgattaccctacccaagacgcccaatac
acaaataatcaagggaagaacattcttttcatgtggggcataaatcacccacccaccgatactgca
cagacaaatctgtacacgagaaccgacacaacaacgagtgtggcaacagaagaaataaataggatc
ttcaaaccattgataggaccaaggcctcttgtcaacggtttgatgggaagaattgattattattgg
tctgtattgaaaccgggtcaaacactgcgaataaaatctgatgggaatctaatagctccatggtat
ggacacattctttcaggagagagccatggaagaatcctgaagactgatttaaaaaggggtagctgc
acagtgcaatgtcagacagagaaaggtggcttaaacacaacactgccattccaaaatgtaagtaag
tatgcatttggaaactgctcaaaatacattggcataaagagtctcaaacttgcagttggtctgagg
aatgtgccttccagatctagtagaggactattcggggccatagcaggattcatagagggaggttgg
tcagggctagttgctggttggtatggattccagcattcaaatgaccaaggggttggtatggcagca
gatagagactcaacccaaaaggcaattgataaaataacatccaaagtgaataacatagtggacaaa
atgaacaagcagtatgaaattattgatcatgaattcagtgaggttgaaactagacttaacatgatc
aataataagattgatgatcaaattcaagatatatgggcatataatgcagaattgctagttctgctt
gagaaccagaaaacactcgatgagcatgatgcaaatgtaaataatctatataataaagtgaagagg
gcattgggtaccaatgcggtggaagatgggaaaggatgtttcgagctataccacaaatgtgatgac
cagtgcatggagacaattcggaacgggacctacaacaggaggaagtatcaagaagaatcaaaatta
gaaaggcagaaaatagagggggtcaagctggaatctgaaggaacttacaaaatcctcaccatttat
tcgactgtcgcctcatctcttgtaattgcaatggggtttgctgccttcttgttctgggcatgtc
aatgggtcttgcagatgcaacatttgtatataa
SEQ ID NO: 11
Name: H9-CNn3
Type: nucleotide
Length: 1683
atggaagtagtatcactaataactatactactagtagtaacagtaagcaatgcagataaaatctgc
atcggctaccaatcaacaaactccacagaaactgtggacacactaacagaaaacaatgtccctgtg
acacatgccaaagaactgctccacacagagcataatgggatgctgtgtgcaacaagcttgggacaa
cctcttattttagacacctgcaccattgaagggctaatctatggcaatccttcttgtgatctatcg
ctggaaggaagagaatggtcctatatcgtcgagagaccatcagctgttaacggattgtgttacccc
gggaatgtagaaaacctagaagagctaaggtcactttttagttctgctaggtcttatcaaagaatc
cagattttcccagacacaatctggaatgtgtcttacgatggaacaagcacagcatgctcaggttca
ttctacagaagcatgagatggttgactcaaaagaacaacgcttaccctatccaagacgcccaatac
acaaataatcaagggaagaacattcttttcatgtggggcataaatcacccacccaccgatactacg
cagagaaatctgtacacgagaaccgacacaacaacgagtgtggcaacagaagaaataaataggatc
ttcaaaccattgataggaccaaggcctcttgtcaacggtttgatgggaagaattgattattattgg
totgtattgaaaccgggtcaaacactgcgaataaaatctgatgggaatctaatagctccatggtat
ggacacattctttcaggagagagccatggaagaatcctgaagactgatttaaaaaggggtagctgc
acagtgcaatgtcagacagagaaaggtggcttaaacacaacactgccattccaaaatgtaagtaag
tatgcatttggaaactgctcaaaatacattggcataaagagtctcaaacttgcagttggtctgagg
aatgtgccttccagatctagtagaggactattcggggccatagcaggattcatagagggaggttgg
tcagggctagttgctggttggtatggattccagcattcaaatgaccaaggggttggtatggcagca
gatagagactcaacccaaaaggcaattgataaaataacatccaaagtgaataacatagtggacaaa
atgaacaagcagtatgaaattattgatcatgaattcagtgaggttgaaactagacttaacatgatc
aataataagattgatgatcaaattcaagatatatgggcatataatgcagaattgctagttctgctt
gagaaccagaaaacactcgatgagcatgatgcaaatgtaaataatctatataataaagtgaagagg
gcattgggtaccaatgcggtggaagatgggaaaggatgtttcgagctataccacaaatgtgatgac
cagtgcatggagacaattcggaacgggacctacaacaggaggaagtatcaagaagaatcaaaatta
gaaaggcagaaaatagagggggtcaagctggaatctgaaggaacttacaaaatcctcaccatttat
tcgactgtcgcctcatctcttgtaattgcaatggggtttgctgccttcttgttctgggccatgtcc
aatgggtcttgcagatgcaacatttgtatataa
SEQ ID NO: 12
Name: H9-CNn4
Type: nucleotide
Length: 1683
atggaagtagtatcactaataactatactactagtagtaacagtaagcaatgcagataaaatctgc
atcggctaccaatcaacaaactccacagaaactgtggacacactaacagaaaacaatgtccctgtg
acacatgccaaagaactgctccacacagagcataatgggatgctgtgtgcaacaagcttgggacaa
cctcttattttagacacctgcaccattgaagggctaatctatggcaatccttcttgtgatctattg
ctgggaggaagagaatggtcctatatcgtcgagagaccatcagctgttaacggattgtgttacccc
gggaatgtagaaaacctagaagagctaaggtcactttttagttctgctaggtcttatcaaagaatc
cagattttcccagacacaatctggaatgtgtcttacgatggaacaagcacagcatgctcaggttca
ttctacagaagcatgagatggttgactcgaaagaacggcgattaccctatccaagacgcccaatac
acaaataatcaagggaagaacattcttttcatgtggggcataaatcacccacccaccgatgatacg
cagagaaatctgtacacgagaaccgacacaacaacgagtgtggcaacagaagaaataaataggatc
ttcaaaccattgataggaccaaggcctcttgtcaacggtttgatgggaagaattgattattattgg
tctgtattgaaaccgggtcaaacactgcgaataaaatctgatgggaatctaatagctccatggtat
ggacacattctttcaggagagagccatggaagaatcctgaagactgatttaaaaaggggtagctgc
acagtgcaatgtcagacagagaaaggtggcttaaacacaacactgccattccaaaatgtaagtaag
tatgcatttggaaactgctcaaaatacattggcataaagagtctcaaacttgcagttggtctgagg
aatgtgccttccagatctagtagaggactattcggggccatagcaggattcatagagggaggttgg
tcagggctagttgctggttggtatggattccagcattcaaatgaccaaggggttggtatggcagca
gatagagactcaacccaaaaggcaattgataaaataacatccaaagtgaataacatagtggacaaa
atgaacaagcagtatgaaattattgatcatgaattcagtgaggttgaaactagacttaacatgatc
aataataagattgatgatcaaattcaagatatatgggcatataatgcagaattgctagttctgctt
gagaaccagaaaacactcgatgagcatgatgcaaatgtaaataatctatataataaagtgaagagg
gcattgggtaccaatgcggtggaagatgggaaaggatgtttcgagctataccacaaatgtgatgac
cagtgcatggagacaattoggaacgggacctacaacaggaggaagtatcaagaagaatcaaaatta
gaaaggcagaaaatagagggggtcaagctggaatctgaaggaacttacaaaatcctcaccatttat
aatgggtcttgcagatgcaacatttgtatataa
SEQ ID NO: 13
Name: H9-CNn5
Type: nucleotide
Length: 1680
atggaagtagtatcactaataactatactactagtagtaacagtaagcaatgcagataaaatctgc
atcggctaccaatcaacaaactccacagaaactgtggacacactaacagaaaacaatgtccctgtg
acacatgccaaagaactgctccacacagagcataatgggatgctgtgtgcaacaagcttgggacaa
cctcttattttagacacctgcaccattgaagggctaatctatggcaatccttcttgtgatctatcg
ctggaaggaagagaatggtcctatatcgtcgagagaccatcagctgttaacggattgtgttacccc
gggaatgtagaaaacctagaagagctaaggtcactttttagttctgctaggtcttatcaaagaatc
cagattttcccagacacaatctggaatgtgtcttacgatggaacaagcacagcatgctcaggttca
ttctacagaagcatgagatggttgactcgaaagaacggcgattaccctatccaagacgcccaatac
acaaataatcaagggaagaacattcttttcatgtggggcataaatcacccacccaccgatgatacg
cagagaaatctgtacacgagaaccgacacaacaacgagtgtggcaacagaagaaataaataggatc
ttcaaaccattgataggaccaaggcctcttgtcaacggtttgatgggaagaattgattattattgg
tctgtattgaaaccgggtcaaacactgcgaataaaatctgatgggaatctaatagctccatggtat
ggacacattctttcaggagagagccatggaagaatcctgaagactgatttaaaaaggggtagctgc
acagtgcaatgtcagacagagaaaggtggcttaaacacaacactgccattccaaaatgtaagtaag
tatgcatttggaaactgctcaaaatacattggcataaagagtctcaaacttgcagttggtctgagg
aatgtgccttccagatctagtagaggactattcggggccatagcaggattcatagagggaggttgg
tcagggctagttgctggttggtatggattccagcattcaaatgaccaaggggttggtatggcagca
gatagagactcaacccaaaaggcaattgataaaataacatccaaagtgaataacatagtggacaaa
atgaacaagcagtatgaaattattgatcatgaattcagtgaggttgaaactagacttaacatgatc
aataataagattgatgatcaaattcaagatatatgggcatataatgcagaattgctagttctgctt
gagaaccagaaaacactcgatgagcatgatgcaaatgtaaataatctatataataaagtgaagagg
gcattgggtaccaatgcggtggaagatgggaaaggatgtttcgagctataccacaaatgtgatgac
cagtgcatggagacaattcggaacgggacctacaacaggaggaagtatcaagaagaatcaaaatta
gaaaggcagaaaatagagggggtcaagctggaatctgaaggaacttacaaaatcctcaccatttcc
tttgccatatcatgctttttgctttgtgttgttctattggggttcattatgtgggcctgccagaat
gggtcttgcagatgcaacatttgtatataa
SEQ ID NO: 14
Name: Newcastle disease virus F gene
Type: nucleotide
Length: 1662
atgggctccagatcttctaccaggatcccagtacctctgatgctgaccgtccgaatcatgttggca
ctgagttgcgtctgtccgaccagctcccttgatggcaggcctcttgcagctgcagggattgtggta
acaggagacaaagcagtcaacatatacacctcatctcagacagggtcaatcataatcaagttactc
ccaaatatgcccaaggataaagaggcgtgtgcaaaagccccattggaagcatacaacaggacattg
actactttgctcaccccccttggtgattctatccgtaggatacaagagtctgtgaccacatccgga
ggagggaaacagggacgtcttataggcgccattatcggtggtgtagctctcggggttgcaaccgct
gcacagataacagcagcctcggctctgatacaagccaatcaaaatgctgccaacatcctccggctc
aaagagagcattgctgcaaccaatgaggctgtgcacgaggtcactgacggattatcacaactagca
gtggcagttgggaagatgcagcaatttgttaatgaccagtttaataaaacagctcaggaattggac
tgtataaaaattacacagcaggttggtgtagaactcaacctgtacctaactgaattgactacagta
ttcgggccacaaatcacttcccctgccttaactcagctgactatccaggcgctttacaatctagct
ggtgggaatatggattacttgttgactaagttaggtgtaggaaacaaccaactcagctcattaatt
ggtagtggcctgattaccggcaaccctatcctgtacgactcacagactcaactcttgggtatacag
gtcaccctaccctcagtcgggaatctaaataatatgcgtgccacctacctggaaaccttgtctgta
agtacaaccaaaggatttgcctcagcacttgtcccaaaagtagtgacacaggttggttccgtgata
gaagagcttgacacctcgtactgtatcgagaccgatttggacctatattgtacaagaatagtgaca
ttccctatgtctcctggtatttattcctgtttgagtggcaatacatctgcttgcatgtattcaaag
actgaaggcgcactcactacgccgtatatgaccctcaaaggctcagttattgccaactgtaagatg
acaacatgtagatgtgcagaccccccgggtatcatatcgcagaattatggagaagctgtgtctcta
atagataggcaatcatgcaatatcttatccttagacgggataactttgaggctcagtggggaattt
gatgcaacttatcaaaagaatatctcaatacaagattctcaagtaatagttacaggcaatcttgac
atctcgactgagcttgggaatgtcaacaactcgataagtaatgctttggataagttagaggaaagc
aacagcaaactagacaaggtcaatgttaaactgaccagcacatccgctcttattacctatatcgtt
ttaactgtcatatctcttgtatgtggtatacttagcctggttctagcatgctacctgatgtacaag
caaaaggcgcaacagaagaccttgttgtggcttgggaataataccctagaccagatgagggccact
acaaaaatgtga
SEQ ID NO: 15
Name: Pec promoter
Type: nucleotide
tgcagagtta ttaatagtaa tcaattacgg ggtcattagt tcatagccca tatatggagy
tccgcgttac ataacttacg gtaaatggcc cgccggctga ccgcccaacg acccccgccc
attgacgtca ataatgacgt atgytcccat agtaacgcca atagggactt tccattgacg
tcaatgggtg gagtayttac ggtaaactgc ccattggcag tacatcaagt gtatcatatg
ccaagtacgc cccctattga cgtcaatgac ggtaaatgga tgcagtattt tgtgcagcga
tgggggcggg gggggggggc gcgcgccagg cggggcgggg cggggcgggg ggcggggcgg
ggcgaggcgg agaggtgcgg cggcagccaa tcagagcggc gcgctccgaa agtttccttt
tatggcgagg cggcggcggc ggcggcccta taaaaagcga agcgcgcggc gggcgggagt
cgctgcgcgc tgccttcgcc ccgtgccccg ctccgccgcc gcctcgcgcc gcccgccccg
gctctgactg accgcgt
SEQ ID NO: 16
Name: murine cytomegalovirus iel promoter
Type: nucleotide
Length: 572
ggccaataag gctgcagtac tgagtcatta gggactttcc aatgggtttt gcccagtaca
taaggtcaat aggggtgaat caacaggaaa gtcccattgg agccaagtac actgagtcaa
tagggacttt ccattgggtt ttgcccagta caaaaggtca atagggggtg agtcaatggg
tttttcccat tattggcacg tacataaggt caataggggt gagtcattgg gtttttccag
ccaatttaat taaaacgcca tgtactttcc caccattgac gtcaatgggc tattgaaact
aatgcaacgt gacctttaaa cggtactttc ccatagctga ttaatgggaa agtaccgttc
tcgagccaat acacgtcaat gggaagtgaa agggcagcca aaacgtaaca ccgccccggt
tttcccctgg aaattccata ttggcacgca ttctattggc tgagctgcgt tctacgtggg
tataagaggc gcgaccagcg tcggtaccgt cgcagtcttc ggtctgacca ccgtagaacg
cagagctcct cgctgcaggc ggccgctcta ga
SEQ ID NO: 17
Name: polyA signal
Type: nucleotide
Length: 87
ctgcaggcgg ccgctctaga gtcgacaata aaagatcttt attttcatta gatctgtgtg
ttggtttttt gtgtggccaa taaggcc
SEQ ID NO: 18
Name: primer
Type: nucleotide
Length: 24
ggggaagtct tccggttaag ggac
SEQ ID NO: 19
Name: primer
Type: nucleotide
Length: 24
ggtgcaattc gtaagaccga tggg
SEQ ID NO: 20
Name: primer
Type: nucleotide
Length: 19
gegcgactcc atacattga
SEQ ID NO: 21
Name: primer
Type: nucleotide
Length: 24
agtccacatg caccccacct aaac

1. A recombinant Herpes Virus of Turkeys (rHVT) which comprises (i) a nucleotide sequence encoding a F protein of Newcastle Disease Virus, or an immunogenic fragment or variant thereof, inserted into a first insertion site of the viral genome; and (ii) a nucleotide sequence encoding an Hemagglutinin (HA) protein of a subtype H9 avian influenza virus, or an immunogenic fragment or variant thereof, inserted into a second insertion site of the viral genome, the first and second insertion sites being located in different non-coding regions of the viral genome selected from the non-coding region between UL45 and UL46, and the non-coding region between SORF3 and US2.

2. The rHVT of claim 1, comprising (i) a nucleotide sequence encoding a F protein of Newcastle Disease Virus, or an immunogenic fragment or variant thereof, inserted into a non-coding region of the viral genome located between UL45 and UL46; and (ii) a nucleotide sequence encoding an Hemagglutinin (HA) protein of a subtype H9 avian influenza virus, or an immunogenic fragment or variant thereof, inserted into a non-coding region of the viral genome located between SORF3 and US2.

3. The rHVT of claim 1, comprising (i) a nucleotide sequence encoding a F protein of Newcastle Disease Virus, or an immunogenic fragment or variant thereof, inserted into a non-coding region of the viral genome located between SORF3 and US2; and (ii) a nucleotide sequence encoding an Hemagglutinin (HA) protein of a subtype H9 avian influenza virus, or an immunogenic fragment or variant thereof, inserted into a non-coding region of the viral genome located between UL45 and UL46.

4. The rHVT of claim 1, wherein the Hemagglutinin (HA) protein is of a subtype H9N2 avian influenza virus.

5. The rHVT of claim 1, wherein the sequence encoding the HA protein is selected from SEQ ID NOs: 8 to 13.

6. The rHVT of claim 1, wherein the sequence encoding the HA protein is under control of a CMV immediate early promoter, preferably a murine or human CMV immediate early promoter.

7. The rHVT of claim 6, wherein the mCMV IE1 promoter comprises a sequence of SEQ ID NO: 15.

8. The rHVT of claim 1, wherein the sequence encoding the F protein is under control of a Pee promoter, preferably wherein the Pee promoter comprises a sequence of SEQ ID NO: 14.

9. A vaccine which comprises a rHVT of claim 1.

10. A method of vaccinating an avian such as a poultry, preferably a chicken comprising administering rHVT of claim 1 to the poultry.

11. A polypeptide comprising, consisting essentially of, or consisting of, a sequence selected from any one of SEQ ID NOs: 2 to 7.

12. A nucleic acid encoding a polypeptide of claim 11, preferably wherein the nucleic acid comprises, consists essentially, or consists of a sequence selected from any one of SEQ ID NOs: 8 to 13.

13. A vector comprising a nucleic acid of claim 12, preferably wherein the vector is a recombinant virus.

14. A recombinant cell comprising a nucleic acid of claim 12.

15. A vaccine which comprises a polypeptide of claim 11.


SEQUENCE LISTING (21 sequences)

SEQ ID NO: 1 - protein, 553 aa, Artificial Sequence
  F protein
MGSRSSTRIPVPLMLTVRIMLALSCVCPTSSLDGRPLAAAGIVVTGDKAVNIYTSSQTGSIIIKLLPNMPKDKEACAKAPLEAYNRTLTTLLTPLGDSIRRIQESVTTSGGGKQGRLIGAIIGGVALGVATAAQITAASALIQANQNAANILRLKESIAATNEAVHEVTDGLSQLAVAVGKMQQFVNDQFNKTAQELDCIKITQQVGVELNLYLTELTTVFGPQITSPALTQLTIQALYNLAGGNMDYLLTKLGVGNNQLSSLIGSGLITGNPILYDSQTQLLGIQVTLPSVGNLNNMRATYLETLSVSTTKGFASALVPKVVTQVGSVIEELDTSYCIETDLDLYCTRIVTFPMSPGIYSCLSGNTSACMYSKTEGALTTPYMTLKGSVIANCKMTTCRCADPPGIISQNYGEAVSLIDRQSCNILSLDGITLRLSGEFDATYQKNISIQDSQVIVTGNLDISTELGNVNNSISNALDKLEESNSKLDKVNVKLTSTSALITYIVLTVISLVCGILSLVLACYLMYKQKAQQKTLLWLGNNTLDQMRATTKM

SEQ ID NO: 2 - protein, 560 aa, Artificial Sequence
  H9-CS
METISLMTILLVVTTSNADKICIGHQSTNSTETVDTLTETNVPVTHAKELLHTEHNGMLCATNLGHPLILDTCTIEGLVYGNPSCDLLLGGREWSYIVERPSAVNGTCYPGNVENLEELRTLFSSSSSYQRIQIFPDTIWNVTYTGTSKSCSDSFYRNMRWLTQKNGVYPVQDAQYTNNRGKDILFVWGIHHPPTDTAQTNLYTRTDTTTSVTTENLDRTFKPLIGPRPLVNGLIGRINYYWSVLKPGQTLRVRSNGNLIAPWFGHVLSGESHGRILKTDLNSGNCVVQCQTEKGGLNSTLPFHNISKYAFGTCPKYIGVKSLKLAIGLRNVPARSSRGLFGAIAGFIEGGWPGLVAGWYGFQHSNDQGVGMAADRDSTQKAVDKITSKVNNIVDKMNKQYEIIDHEFSEVETRLNMINNKIDDQIQDVWAYNAELLVLLENQKTLDEHDANVNNLYNKVKRALGSNAIEDGKGCFELYHKCDNQCMETIRNGTYNRRKYKEESRLERQKIEGVKLESEGTYKILTIYSTVASSLVLAMVFAAFLFWAMSNGSCRCNICI

SEQ ID NO: 3 - protein, 560 aa, Artificial Sequence
  H9-CNn1
MEVVSLITILLVVTVSNADKICIGYQSTNSTETVDTLTENNVPVTHAKELLHTEHNGMLCATSLGQPLILDTCTIEGLIYGNPSCDLSLEGREWSYIVERPSAVNGLCYPGNVENLEELRSLFSSARSYQRIQIFPDTIWNVSYDGTSTACSGSFYRSMRWLTRKNGDYPIQDAQYTNNQGKNILFMWGINHPPTDDTQRNLYTRTDTTTSVATEEINRIFKPLIGPRPLVNGLMGRIDYYWSVLKPGQTLRIKSDGNLIAPWYGHILSGESHGRILKTDLKRGSCTVQCQTEKGGLNTTLPFQNVSKYAFGNCSKYIGIKSLKLAVGLRNVPSRSSRGLFGAIAGFIEGGWSGLVAGWYGFQHSNDQGVGMAADRDSTQKAIDKITSKVNNIVDKMNKQYEIIDHEFSEVETRLNMINNKIDDQIQDIWAYNAELLVLLENQKTLDEHDANVNNLYNKVKRALGTNAVEDGKGCFELYHKCDDQCMETIRNGTYNRRKYQEESKLERQKIEGVKLESEGTYKILTIYSTVASSLVIAMGFAAFLFWAMSNGSCRCNICI

SEQ ID NO: 4 - protein, 560 aa, Artificial Sequence
  H9-CNn2
MEVVSLITILLVVTVSNADKICIGYQSTNSTETVDTLTENNVPVTHAKELLHTEHNGMLCATSLGQPLILDTCTIEGLIYGNPSCDLSLEGREWSYIVERPSAVNGLCYPGNVENLEELRSLFSSARSYQRIQIFPDTIWNVSYDGTSTACSGSFYRSMRWLTRKNGDYPTQDAQYTNNQGKNILFMWGINHPPTDTAQTNLYTRTDTTTSVATEEINRIFKPLIGPRPLVNGLMGRIDYYWSVLKPGQTLRIKSDGNLIAPWYGHILSGESHGRILKTDLKRGSCTVQCQTEKGGLNTTLPFQNVSKYAFGNCSKYIGIKSLKLAVGLRNVPSRSSRGLFGAIAGFIEGGWSGLVAGWYGFQHSNDQGVGMAADRDSTQKAIDKITSKVNNIVDKMNKQYEIIDHEFSEVETRLNMINNKIDDQIQDIWAYNAELLVLLENQKTLDEHDANVNNLYNKVKRALGTNAVEDGKGCFELYHKCDDQCMETIRNGTYNRRKYQEESKLERQKIEGVKLESEGTYKILTIYSTVASSLVIAMGFAAFLFWAMSNGSCRCNICI

SEQ ID NO: 5 - protein, 560 aa, Artificial Sequence
  H9-CNn3
MEVVSLITILLVVTVSNADKICIGYQSTNSTETVDTLTENNVPVTHAKELLHTEHNGMLCATSLGQPLILDTCTIEGLIYGNPSCDLSLEGREWSYIVERPSAVNGLCYPGNVENLEELRSLFSSARSYQRIQIFPDTIWNVSYDGTSTACSGSFYRSMRWLTQKNNAYPIQDAQYTNNQGKNILFMWGINHPPTDTTQRNLYTRTDTTTSVATEEINRIFKPLIGPRPLVNGLMGRIDYYWSVLKPGQTLRIKSDGNLIAPWYGHILSGESHGRILKTDLKRGSCTVQCQTEKGGLNTTLPFQNVSKYAFGNCSKYIGIKSLKLAVGLRNVPSRSSRGLFGAIAGFIEGGWSGLVAGWYGFQHSNDQGVGMAADRDSTQKAIDKITSKVNNIVDKMNKQYEIIDHEFSEVETRLNMINNKIDDQIQDIWAYNAELLVLLENQKTLDEHDANVNNLYNKVKRALGTNAVEDGKGCFELYHKCDDQCMETIRNGTYNRRKYQEESKLERQKIEGVKLESEGTYKILTIYSTVASSLVIAMGFAAFLFWAMSNGSCRCNICI

SEQ ID NO: 6 - protein, 560 aa, Artificial Sequence
  H9-CNn4
MEVVSLITILLVVTVSNADKICIGYQSTNSTETVDTLTENNVPVTHAKELLHTEHNGMLCATSLGQPLILDTCTIEGLIYGNPSCDLLLGGREWSYIVERPSAVNGLCYPGNVENLEELRSLFSSARSYQRIQIFPDTIWNVSYDGTSTACSGSFYRSMRWLTRKNGDYPIQDAQYTNNQGKNILFMWGINHPPTDDTQRNLYTRTDTTTSVATEEINRIFKPLIGPRPLVNGLMGRIDYYWSVLKPGQTLRIKSDGNLIAPWYGHILSGESHGRILKTDLKRGSCTVQCQTEKGGLNTTLPFQNVSKYAFGNCSKYIGIKSLKLAVGLRNVPSRSSRGLFGAIAGFIEGGWSGLVAGWYGFQHSNDQGVGMAADRDSTQKAIDKITSKVNNIVDKMNKQYEIIDHEFSEVETRLNMINNKIDDQIQDIWAYNAELLVLLENQKTLDEHDANVNNLYNKVKRALGTNAVEDGKGCFELYHKCDDQCMETIRNGTYNRRKYQEESKLERQKIEGVKLESEGTYKILTIYSTVASSLVIAMGFAAFLFWAMSNGSCRCNICI

SEQ ID NO: 7 - protein, 559 aa, Artificial Sequence
  H9-CNn5
MEVVSLITILLVVTVSNADKICIGYQSTNSTETVDTLTENNVPVTHAKELLHTEHNGMLCATSLGQPLILDTCTIEGLIYGNPSCDLSLEGREWSYIVERPSAVNGLCYPGNVENLEELRSLFSSARSYQRIQIFPDTIWNVSYDGTSTACSGSFYRSMRWLTRKNGDYPIQDAQYTNNQGKNILFMWGINHPPTDDTQRNLYTRTDTTTSVATEEINRIFKPLIGPRPLVNGLMGRIDYYWSVLKPGQTLRIKSDGNLIAPWYGHILSGESHGRILKTDLKRGSCTVQCQTEKGGLNTTLPFQNVSKYAFGNCSKYIGIKSLKLAVGLRNVPSRSSRGLFGAIAGFIEGGWSGLVAGWYGFQHSNDQGVGMAADRDSTQKAIDKITSKVNNIVDKMNKQYEIIDHEFSEVETRLNMINNKIDDQIQDIWAYNAELLVLLENQKTLDEHDANVNNLYNKVKRALGTNAVEDGKGCFELYHKCDDQCMETIRNGTYNRRKYQEESKLERQKIEGVKLESEGTYKILTISFAISCFLLCVVLLGFIMWACQNGSCRCNICI

SEQ ID NO: 8 - DNA, 1683 bp, Artificial Sequence
  H9-CS
atggaaacaatatcactgatgactatactactagtagtaacaacaagcaatgcagataaaatctgcattggccaccagtcaacaaattccacagaaactgtggacacgctaacagaaactaatgttcctgtgacacatgccaaagaattgctccacacagagcacaatggaatgctgtgtgcaacaaatctgggacatcccctcatcctagacacatgcactatcgaaggacttgtctatggtaacccttcttgtgatctgttgttggggggaagagaatggtcctacatcgttgaaagaccatcagctgtaaatggaacgtgttaccctgggaatgtggaaaacttagaggaactcagaacactctttagttcctctagttcctaccaaagaatccaaatattcccagacacaatctggaatgtgacttacactggaacaagcaaatcatgttcagattcattctacaggaatatgagatggctgactcaaaagaacggggtttatcctgttcaagacgcccaatacacaaataatcggggaaaagacattcttttcgtgtggggtatacatcatccacccaccgatactgcacagacgaatttgtacacaagaaccgacacaacaacaagtgtaacaacagaaaatttagatagaaccttcaaaccattgatagggccaaggccccttgtcaatggtctgattggaagaattaattattattggtcagtactaaaaccaggccagacattgcgagtaagatccaatgggaatttgattgctccatggtttggacacgttctctcaggggagagccatggtagaatcctgaaaactgatttaaacagtggtaattgtgtagtgcaatgtcagactgaaaaaggtggcctaaacagtacattgcctttccacaatatcagtaaatatgcatttgggacttgccccaaatatattggagtcaagagtctcaaactggcaatcggtctgagaaacgtgcctgctaggtcaagtagaggactatttggagccatagctggattcatagaaggaggttggccagggctagttgccggttggtatggtttccagcattcaaatgatcaaggggttggtatggcagcagatagggattcaactcaaaaggcagttgataaaataacatccaaggtgaataatatagtggacaagatgaacaagcaatatgaaataattgatcatgaattcagtgaggttgaaactagactcaatatgatcaataataagattgatgaccaaatacaagatgtatgggcatataatgcagagttgctagtactacttgagaaccagaaaacactcgatgagcatgacgcaaacgtgaacaacctatataacaaggtgaaaagggccttgggctccaatgcgattgaagatgggaagggctgtttcgagctataccacaaatgtgataaccaatgcatggaaactattcggaatgggacctataataggaggaagtacaaagaggaatcaagactagaaagacagaaaatagagggggtcaaactggaatctgaggggacttacaaaatacttaccatttattcgactgtcgcctcatctcttgtacttgcaatggtgtttgctgccttcttattctgggccatgtccaatggatcatgcaggtgcaacatctgtatataa

SEQ ID NO: 9 - DNA, 1683 bp, Artificial Sequence
  H9-CNn1
atggaagtagtatcactaataactatactactagtagtaacagtaagcaatgcagataaaatctgcatcggctaccaatcaacaaactccacagaaactgtggacacactaacagaaaacaatgtccctgtgacacatgccaaagaactgctccacacagagcataatgggatgctgtgtgcaacaagcttgggacaacctcttattttagacacctgcaccattgaagggctaatctatggcaatccttcttgtgatctatcgctggaaggaagagaatggtcctatatcgtcgagagaccatcagctgttaacggattgtgttaccccgggaatgtagaaaacctagaagagctaaggtcactttttagttctgctaggtcttatcaaagaatccagattttcccagacacaatctggaatgtgtcttacgatggaacaagcacagcatgctcaggttcattctacagaagcatgagatggttgactcgaaagaacggcgattaccctatccaagacgcccaatacacaaataatcaagggaagaacattcttttcatgtggggcataaatcacccacccaccgatgatacgcagagaaatctgtacacgagaaccgacacaacaacgagtgtggcaacagaagaaataaataggatcttcaaaccattgataggaccaaggcctcttgtcaacggtttgatgggaagaattgattattattggtctgtattgaaaccgggtcaaacactgcgaataaaatctgatgggaatctaatagctccatggtatggacacattctttcaggagagagccatggaagaatcctgaagactgatttaaaaaggggtagctgcacagtgcaatgtcagacagagaaaggtggcttaaacacaacactgccattccaaaatgtaagtaagtatgcatttggaaactgctcaaaatacattggcataaagagtctcaaacttgcagttggtctgaggaatgtgccttccagatctagtagaggactattcggggccatagcaggattcatagagggaggttggtcagggctagttgctggttggtatggattccagcattcaaatgaccaaggggttggtatggcagcagatagagactcaacccaaaaggcaattgataaaataacatccaaagtgaataacatagtggacaaaatgaacaagcagtatgaaattattgatcatgaattcagtgaggttgaaactagacttaacatgatcaataataagattgatgatcaaattcaagatatatgggcatataatgcagaattgctagttctgcttgagaaccagaaaacactcgatgagcatgatgcaaatgtaaataatctatataataaagtgaagagggcattgggtaccaatgcggtggaagatgggaaaggatgtttcgagctataccacaaatgtgatgaccagtgcatggagacaattcggaacgggacctacaacaggaggaagtatcaagaagaatcaaaattagaaaggcagaaaatagagggggtcaagctggaatctgaaggaacttacaaaatcctcaccatttattcgactgtcgcctcatctcttgtaattgcaatggggtttgctgccttcttgttctgggccatgtccaatgggtcttgcagatgcaacatttgtatataa

SEQ ID NO: 10 - DNA, 1683 bp, Artificial Sequence
  H9-CNn2
atggaagtagtatcactaataactatactactagtagtaacagtaagcaatgcagataaaatctgcatcggctaccaatcaacaaactccacagaaactgtggacacactaacagaaaacaatgtccctgtgacacatgccaaagaactgctccacacagagcataatgggatgctgtgtgcaacaagcttgggacaacctcttattttagacacctgcaccattgaagggctaatctatggcaatccttcttgtgatctatcgctggaaggaagagaatggtcctatatcgtcgagagaccatcagctgttaacggattgtgttaccccgggaatgtagaaaacctagaagagctaaggtcactttttagttctgctaggtcttatcaaagaatccagattttcccagacacaatctggaatgtgtcttacgatggaacaagcacagcatgctcaggttcattctacagaagcatgagatggttgactcgaaagaacggcgattaccctacccaagacgcccaatacacaaataatcaagggaagaacattcttttcatgtggggcataaatcacccacccaccgatactgcacagacaaatctgtacacgagaaccgacacaacaacgagtgtggcaacagaagaaataaataggatcttcaaaccattgataggaccaaggcctcttgtcaacggtttgatgggaagaattgattattattggtctgtattgaaaccgggtcaaacactgcgaataaaatctgatgggaatctaatagctccatggtatggacacattctttcaggagagagccatggaagaatcctgaagactgatttaaaaaggggtagctgcacagtgcaatgtcagacagagaaaggtggcttaaacacaacactgccattccaaaatgtaagtaagtatgcatttggaaactgctcaaaatacattggcataaagagtctcaaacttgcagttggtctgaggaatgtgccttccagatctagtagaggactattcggggccatagcaggattcatagagggaggttggtcagggctagttgctggttggtatggattccagcattcaaatgaccaaggggttggtatggcagcagatagagactcaacccaaaaggcaattgataaaataacatccaaagtgaataacatagtggacaaaatgaacaagcagtatgaaattattgatcatgaattcagtgaggttgaaactagacttaacatgatcaataataagattgatgatcaaattcaagatatatgggcatataatgcagaattgctagttctgcttgagaaccagaaaacactcgatgagcatgatgcaaatgtaaataatctatataataaagtgaagagggcattgggtaccaatgcggtggaagatgggaaaggatgtttcgagctataccacaaatgtgatgaccagtgcatggagacaattcggaacgggacctacaacaggaggaagtatcaagaagaatcaaaattagaaaggcagaaaatagagggggtcaagctggaatctgaaggaacttacaaaatcctcaccatttattcgactgtcgcctcatctcttgtaattgcaatggggtttgctgccttcttgttctgggccatgtccaatgggtcttgcagatgcaacatttgtatataa

SEQ ID NO: 11 - DNA, 1683 bp, Artificial Sequence
  H9-CNn3
atggaagtagtatcactaataactatactactagtagtaacagtaagcaatgcagataaaatctgcatcggctaccaatcaacaaactccacagaaactgtggacacactaacagaaaacaatgtccctgtgacacatgccaaagaactgctccacacagagcataatgggatgctgtgtgcaacaagcttgggacaacctcttattttagacacctgcaccattgaagggctaatctatggcaatccttcttgtgatctatcgctggaaggaagagaatggtcctatatcgtcgagagaccatcagctgttaacggattgtgttaccccgggaatgtagaaaacctagaagagctaaggtcactttttagttctgctaggtcttatcaaagaatccagattttcccagacacaatctggaatgtgtcttacgatggaacaagcacagcatgctcaggttcattctacagaagcatgagatggttgactcaaaagaacaacgcttaccctatccaagacgcccaatacacaaataatcaagggaagaacattcttttcatgtggggcataaatcacccacccaccgatactacgcagagaaatctgtacacgagaaccgacacaacaacgagtgtggcaacagaagaaataaataggatcttcaaaccattgataggaccaaggcctcttgtcaacggtttgatgggaagaattgattattattggtctgtattgaaaccgggtcaaacactgcgaataaaatctgatgggaatctaatagctccatggtatggacacattctttcaggagagagccatggaagaatcctgaagactgatttaaaaaggggtagctgcacagtgcaatgtcagacagagaaaggtggcttaaacacaacactgccattccaaaatgtaagtaagtatgcatttggaaactgctcaaaatacattggcataaagagtctcaaacttgcagttggtctgaggaatgtgccttccagatctagtagaggactattcggggccatagcaggattcatagagggaggttggtcagggctagttgctggttggtatggattccagcattcaaatgaccaaggggttggtatggcagcagatagagactcaacccaaaaggcaattgataaaataacatccaaagtgaataacatagtggacaaaatgaacaagcagtatgaaattattgatcatgaattcagtgaggttgaaactagacttaacatgatcaataataagattgatgatcaaattcaagatatatgggcatataatgcagaattgctagttctgcttgagaaccagaaaacactcgatgagcatgatgcaaatgtaaataatctatataataaagtgaagagggcattgggtaccaatgcggtggaagatgggaaaggatgtttcgagctataccacaaatgtgatgaccagtgcatggagacaattcggaacgggacctacaacaggaggaagtatcaagaagaatcaaaattagaaaggcagaaaatagagggggtcaagctggaatctgaaggaacttacaaaatcctcaccatttattcgactgtcgcctcatctcttgtaattgcaatggggtttgctgccttcttgttctgggccatgtccaatgggtcttgcagatgcaacatttgtatataa

SEQ ID NO: 12 - DNA, 1683 bp, Artificial Sequence
  H9-CNn4
atggaagtagtatcactaataactatactactagtagtaacagtaagcaatgcagataaaatctgcatcggctaccaatcaacaaactccacagaaactgtggacacactaacagaaaacaatgtccctgtgacacatgccaaagaactgctccacacagagcataatgggatgctgtgtgcaacaagcttgggacaacctcttattttagacacctgcaccattgaagggctaatctatggcaatccttcttgtgatctattgctgggaggaagagaatggtcctatatcgtcgagagaccatcagctgttaacggattgtgttaccccgggaatgtagaaaacctagaagagctaaggtcactttttagttctgctaggtcttatcaaagaatccagattttcccagacacaatctggaatgtgtcttacgatggaacaagcacagcatgctcaggttcattctacagaagcatgagatggttgactcgaaagaacggcgattaccctatccaagacgcccaatacacaaataatcaagggaagaacattcttttcatgtggggcataaatcacccacccaccgatgatacgcagagaaatctgtacacgagaaccgacacaacaacgagtgtggcaacagaagaaataaataggatcttcaaaccattgataggaccaaggcctcttgtcaacggtttgatgggaagaattgattattattggtctgtattgaaaccgggtcaaacactgcgaataaaatctgatgggaatctaatagctccatggtatggacacattctttcaggagagagccatggaagaatcctgaagactgatttaaaaaggggtagctgcacagtgcaatgtcagacagagaaaggtggcttaaacacaacactgccattccaaaatgtaagtaagtatgcatttggaaactgctcaaaatacattggcataaagagtctcaaacttgcagttggtctgaggaatgtgccttccagatctagtagaggactattcggggccatagcaggattcatagagggaggttggtcagggctagttgctggttggtatggattccagcattcaaatgaccaaggggttggtatggcagcagatagagactcaacccaaaaggcaattgataaaataacatccaaagtgaataacatagtggacaaaatgaacaagcagtatgaaattattgatcatgaattcagtgaggttgaaactagacttaacatgatcaataataagattgatgatcaaattcaagatatatgggcatataatgcagaattgctagttctgcttgagaaccagaaaacactcgatgagcatgatgcaaatgtaaataatctatataataaagtgaagagggcattgggtaccaatgcggtggaagatgggaaaggatgtttcgagctataccacaaatgtgatgaccagtgcatggagacaattcggaacgggacctacaacaggaggaagtatcaagaagaatcaaaattagaaaggcagaaaatagagggggtcaagctggaatctgaaggaacttacaaaatcctcaccatttattcgactgtcgcctcatctcttgtaattgcaatggggtttgctgccttcttgttctgggccatgtccaatgggtcttgcagatgcaacatttgtatataa

SEQ ID NO: 13 - DNA, 1680 bp, Artificial Sequence
  H9-CNn5
atggaagtagtatcactaataactatactactagtagtaacagtaagcaatgcagataaaatctgcatcggctaccaatcaacaaactccacagaaactgtggacacactaacagaaaacaatgtccctgtgacacatgccaaagaactgctccacacagagcataatgggatgctgtgtgcaacaagcttgggacaacctcttattttagacacctgcaccattgaagggctaatctatggcaatccttcttgtgatctatcgctggaaggaagagaatggtcctatatcgtcgagagaccatcagctgttaacggattgtgttaccccgggaatgtagaaaacctagaagagctaaggtcactttttagttctgctaggtcttatcaaagaatccagattttcccagacacaatctggaatgtgtcttacgatggaacaagcacagcatgctcaggttcattctacagaagcatgagatggttgactcgaaagaacggcgattaccctatccaagacgcccaatacacaaataatcaagggaagaacattcttttcatgtggggcataaatcacccacccaccgatgatacgcagagaaatctgtacacgagaaccgacacaacaacgagtgtggcaacagaagaaataaataggatcttcaaaccattgataggaccaaggcctcttgtcaacggtttgatgggaagaattgattattattggtctgtattgaaaccgggtcaaacactgcgaataaaatctgatgggaatctaatagctccatggtatggacacattctttcaggagagagccatggaagaatcctgaagactgatttaaaaaggggtagctgcacagtgcaatgtcagacagagaaaggtggcttaaacacaacactgccattccaaaatgtaagtaagtatgcatttggaaactgctcaaaatacattggcataaagagtctcaaacttgcagttggtctgaggaatgtgccttccagatctagtagaggactattcggggccatagcaggattcatagagggaggttggtcagggctagttgctggttggtatggattccagcattcaaatgaccaaggggttggtatggcagcagatagagactcaacccaaaaggcaattgataaaataacatccaaagtgaataacatagtggacaaaatgaacaagcagtatgaaattattgatcatgaattcagtgaggttgaaactagacttaacatgatcaataataagattgatgatcaaattcaagatatatgggcatataatgcagaattgctagttctgcttgagaaccagaaaacactcgatgagcatgatgcaaatgtaaataatctatataataaagtgaagagggcattgggtaccaatgcggtggaagatgggaaaggatgtttcgagctataccacaaatgtgatgaccagtgcatggagacaattcggaacgggacctacaacaggaggaagtatcaagaagaatcaaaattagaaaggcagaaaatagagggggtcaagctggaatctgaaggaacttacaaaatcctcaccatttcctttgccatatcatgctttttgctttgtgttgttctattggggttcattatgtgggcctgccagaatgggtcttgcagatgcaacatttgtatataa

SEQ ID NO: 14 - DNA, 1662 bp, Artificial Sequence
  F gene
atgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtga

SEQ ID NO: 15 - DNA, 557 bp, Artificial Sequence
  Pec promoter
tgcagagttattaatagtaatcaattacggggtcattagttcatagcccatatatggagytccgcgttacataacttacggtaaatggcccgccggctgaccgcccaacgacccccgcccattgacgtcaataatgacgtatgytcccatagtaacgccaatagggactttccattgacgtcaatgggtggagtayttacggtaaactgcccattggcagtacatcaagtgtatcatatgccaagtacgccccctattgacgtcaatgacggtaaatggatgcagtattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgt

SEQ ID NO: 16 - DNA, 572 bp, Artificial Sequence
  mcmv-ie1 promoter
ggccaataaggctgcagtactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgctctaga

SEQ ID NO: 17 - DNA, 87 bp, Artificial Sequence
  polyA signal
ctgcaggcggccgctctagagtcgacaataaaagatctttattttcattagatctgtgtgttggttttttgtgtggccaataaggcc

SEQ ID NO: 18 - DNA, 24 bp, Artificial Sequence
  Primer
ggggaagtcttccggttaagggac

SEQ ID NO: 19 - DNA, 24 bp, Artificial Sequence
  Primer
ggtgcaattcgtaagaccgatggg

SEQ ID NO: 20 - DNA, 19 bp, Artificial Sequence
  Primer
gcgcgactccatacattga

SEQ ID NO: 21 - DNA, 24 bp, Artificial Sequence
  Primer
agtccacatgcaccccacctaaac